(19) United States
(12) Patent Application Publication
(10) Pub. No.: US 2020/0178811 A1
(43) Pub. Date: Jun. 11, 2020

(54) PERIODONTAL DISEASE EXAMINATION APPARATUS, OPTICAL WAVEGUIDE ARRAY, AND MOUTHPIECE

(71) Applicant: Tanita Corporation, Tokyo (JP)

(72) Inventor: Mikio SHINDO, Tokyo (JP)

(21) Appl. No.: 16/751,035

(22) Filed: Jan. 23, 2020

Related U.S. Application Data
(63) Continuation of application No. PCT/JP2018/024101, filed on Jun. 26, 2018.

(30) Foreign Application Priority Data
Jul. 28, 2017 (JP) 2017-146476

(51) Int. Cl. A61B 5/00; A61C 19/04; A61B 1/07

(52) U.S. Cl. CPC A61B 5/0088; A61B 5/4552; A61C 19/043; A61B 5/682; A61B 2562/0233; A61B 2576/02; A61B 2562/046; A61B 2562/043; A61B 1/07

(57) ABSTRACT

Loss of light is reduced in an examination for periodontal disease. A plurality of optical fibers are each held by holder so as to be freely movable independently along the direction of the optical axis. Since the optical fibers are each freely movable independently along the direction of the optical axis, light-emitting end faces of the optical fibers can be brought into close contact with a tooth or gum. Light reflected from the tooth and gum can be made to impinge on the optical fibers with reduced loss.

### CROSS-REFERENCE TO RELATED APPLICATIONS

This application is a Continuation of PCT International Application No. PCT JP2018/024101 filed on Jun. 26, 2018, which claims priority under 35 U.S.C. § 119(a) to Japanese Patent Application No. 2017-146476 filed Jul. 28, 2017. Each of the above application(s) is hereby expressly incorporated by reference, in its entirety, into the present application.

### TECHNICAL FIELD

This invention relates to a periodontal disease examination apparatus, optical waveguide array and mouthpiece.

### BACKGROUND ART

Measurement of the depth of a periodontal pocket is carried out as one example of an examination for periodontal disease. In general, the depth of a periodontal pocket is measured visually as by a dentist inserting a rod-like measuring instrument referred to as a “pocket probe” into the periodontal pocket. However, there are occasions where the result of measurement is not necessarily accurate owing to the level of skill of the dentist or the like, the angle of insertion of the pocket probe and visual error, etc. Further, there is concern that, owing to bleeding from the gums at the time of examination, affected parts free of periodontal disease will become infected with periodontal disease. For these reasons, consideration has been given to the measurement of periodontal pocket depth non-invasively using an optical coherence tomographic diagnostic apparatus (Patent Documents 1, 2). Further, consideration has also been given to an OCT (Optical Coherence Tomography) apparatus in which 48 optical fibers are fixed by an aligning jig and the target is irradiated with light (Patent Document 3). Prior-Art Documents:

### PATENT DOCUMENTS

Patent Document 1: Japanese Patent Application Laid-Open No. 2009-131313
Patent Document 2: Japanese Patent Application Laid-Open No. 2009-148337
Patent Document 3: Japanese Patent Application Laid-Open No. 2011-47814

The surface of teeth and gums takes on a complicated shape and generally is not planar. In a case where the depth of a periodontal pocket is measured non-invasively using the OCT apparatus described in Patent Document 3, often the light-emitting end faces of all of the optical fibers cannot be brought into close contact with teeth and gums owing to the fact that the optical waveguides are fixed by the aligning jig. As a consequence, all of the light reflected from teeth or gums cannot be returned to the optical fibers, and loss may occur.

### DISCLOSURE OF THE INVENTION

An object of the present invention is to reduce loss of light reflected from the surface of teeth and gums.

A periodontal disease examination apparatus according to a first aspect of the present invention is characterized by comprising: an optical divider for splitting low-interference light into measuring light and reference light; a first optical waveguide on which the measuring light split off by the optical divider is incident; an optical waveguide array in which a plurality of second optical waveguides are arrayed in at least a single row; a holder formed of a flexible member for holding the optical waveguide array such that each of the plurality of second optical waveguides is freely movable independently along the direction of its own optical axis; a first control mechanism for controlling the measuring light, which is emitted from the first optical waveguide, the first optical waveguide, and at least one of the second optical waveguides in such a manner that the measuring light emitted from the first optical waveguide impinges successively on each of the second optical waveguides constituting the optical waveguide array; a photodetector for detecting reflected light and outputting an interference signal, the reflected light being reflected measuring light which is reflected from a gum or tooth owing to irradiation of the gum or tooth with the measuring light emitted from said optical waveguide array and reflected reference light which is split off by said optical divider and reflected by a reference surface; and a periodontal pocket data generating device (periodontal pocket data generating means)(processor circuitry) for generating data regarding depth of a periodontal pocket based on the interference signal output from the photodetector.

The optical waveguides include optical fibers, light guides, optical transmission circuits, optical transmission devices, light guide plates, light guide bodies and light guide members or the like. As long as the optical waveguide is a light guide member or light transmitting member, any will suffice irrespective of what it is called.

Preferably, the holder holds each second optical waveguide constituting the optical waveguide array such that the tip of each second optical waveguide constituting the optical waveguide array is protrudable from the tip of the holder but incapable of receding into the tip of the holder.

For example, the holder has a space portion; each second optical waveguide constituting the optical waveguide array passes through the space portion; and the diameter of the tip of each second optical waveguide constituting the optical waveguide array is larger than the diameter of the portion of the second optical waveguide other than the tip thereof and larger than the diameter of the space portion.

The holder may fix the tip of each second optical waveguide constituting the optical waveguide array to the tip of the holder.

The first control mechanism is a first adjusting mechanism for adjusting at least one of: position of a measuring-light-emitting end face of the first optical waveguide, and positions of measuring-light-incident end faces of the second optical waveguides, in such a manner that the measuring-light-emitting end face of the first optical waveguide successively opposes the measuring-light-incident end faces of the second optical waveguides constituting the optical waveguide array.

The apparatus may further comprise a parallelizing element, which is for parallelizing the measuring light, provided on the optical path of the measuring light between the measuring-light-incident end faces of the second optical waveguides and the measuring-light-emitting end face of the first optical waveguide.

The optical waveguide array includes a first optical waveguide array and a second optical waveguide array; the first optical waveguide array includes a plurality of optical waveguides arrayed in at least one row, each of the plurality of these optical waveguides receiving incident light from its measuring-light-incident end face emitted from the first optical waveguide and emitting the measuring light from its measuring-light-emitting end face; the second optical waveguide array includes a plurality of optical waveguides arrayed in at least one row and of a number greater than the number of the plurality of optical waveguides included in the first optical waveguide array, each of the plurality of these optical waveguides receiving incident light from its measuring light-incident-end face emitted from an optical waveguide included in the first optical waveguide array and emitting the measuring light from its measuring-light-emitting end face; the holder holds the plurality of optical waveguides such that each of the plurality of optical waveguides included in the second optical waveguide array is freely movable independently along the direction of its own optical axis; and the apparatus further comprises a second control mechanism for controlling at least one of: optical waveguides included in the first optical waveguide array, and optical waveguides included in the second optical waveguide array, in such a manner that the measuring light emitted from an optical waveguide included in the first optical waveguide array impinges successively on the plurality of optical waveguides included in the second optical waveguide array.

The apparatus further comprises a parallelizing element, which is for parallelizing the measuring light, provided on the optical path of the measuring light between at least one of the following: between the measuring-light-incident end faces of the optical waveguides included in the first optical waveguide array and the measuring-light-emitting end face of the first optical waveguide, and between the measuring-light-incident end faces of the optical waveguides included in the second optical waveguide array and the measuring-light-emitting end faces of the optical waveguides included in the first optical waveguide array.

For example, the first control mechanism is a deflecting mechanism for deflecting measuring light emitted from the first optical waveguide and guiding the measuring light successively to each of the second optical waveguides constituting the optical waveguide array.

A coupling member for separably coupling the optical waveguide array may be provided between the tip of the optical waveguide array and the base end of the optical waveguide array.

The apparatus is such that the holder for holding the optical waveguide fiber array is a mouthpiece for being placed in close contact with the surface portion of teeth at a gum boundary and with a portion of the gums; the light-emitting end faces of the plurality of second optical waveguides being exposed at a surface of close contact of the mouthpiece in close contact with the surface portion of the teeth and the portion of the gums.

An optical waveguide array according to a second aspect of the present invention is characterized by having a plurality of optical waveguides arrayed in at least a single row, wherein out of measuring light and reference light split by an optical divider, the measuring light impinges successively on the optical waveguides, each of the optical waveguides being held freely movable independently along the direction of its own optical axis by a holder comprising a flexible material.

A third aspect of the present invention is a mouthpiece brought into close contact with a surface portion of teeth at a gum boundary and with a portion of the gums and comprising a flexible material, the mouthpiece holding a plurality of optical waveguides such that light-emitting end faces of the plurality of optical waveguides arrayed in at least a single row are exposed at a surface of close contact where said mouthpiece is in close contact with the surface portion of the teeth and the portion of the gums.

In accordance with the first aspect of the present invention, the optical waveguide array is such that the plurality of second optical waveguides are arrayed in at least a single row and are held by the holder, which comprises a flexible material, in such a manner that each of the plurality of second optical waveguides is freely movable independently along the direction of its own optical axis. The plurality of second optical waveguides are not fixed and each is held freely movable independently along the direction of its own optical axis. This makes it possible for the tip face of each of the second optical waveguides to be brought into close contact with a tooth or gum.

When measuring light emitted from the first optical waveguide impinges successively on each of the second optical waveguides constituting the optical waveguide array, measuring light emitted from the tip face of each of the second optical waveguides is reflected at a tooth or gum and substantially all of the reflected light returns to this tip face of this second optical waveguide. Data regarding the depth of a periodontal pocket can be generated based on interference signals obtained from reflected measuring light and reflected reference light. Since loss of reflected light from a tooth and gum can be reduced, the data regarding a periodontal pocket can be generated more accurately.

The second aspect of the present invention is an optical waveguide array used in the periodontal disease examination apparatus according to the first aspect of the present invention. By using the optical waveguide array according to the second aspect of the present invention in the periodontal disease examination apparatus, loss of reflected light from a tooth and gum can be reduced. This means that the data regarding a periodontal pocket can be generated more accurately.

The third aspect of the present invention is a mouthpiece used in the periodontal disease examination apparatus according to the first aspect of the present invention. By using the mouthpiece according to the third aspect of the present invention in the periodontal disease examination apparatus, data regarding the depths of multiple periodontal pockets is obtained in a comparatively short period of time.

### BRIEF DESCRIPTION OF THE DRAWINGS

FIG. 1 is a block diagram illustrating the construction of a periodontal disease examination apparatus;

FIG. 2 illustrates the construction of a deflecting unit;

FIG. 3 illustrates the construction of the deflecting unit;

FIG. 4 is a perspective view of an examination probe;

FIG. 5 is a sectional view taken along line V-V of FIG. 4;

FIG. 6 is a sectional view taken along line VI-VI of FIG. 4;

FIG. 7 illustrates the manner in which a contact portion is brought into contact with a gum and a tooth;

FIG. 8 illustrates the manner in which a contact portion is brought into contact with a gum and a tooth;

FIG. 9 illustrates the manner in which a gum and a tooth are irradiated with measuring light;

FIG. 10A to FIG. 10E are examples of interference signals;

FIG. 11 is an example of optical tomographic images of periodontal pocket;

FIG. 12 is an example of a deflecting unit;

FIG. 13 is an example of a deflecting unit;

FIG. 14 is a perspective view of a stepping motor or the like;

FIG. 15A is a sectional view of a contact portion in a state in which optical fibers have not been inserted, and FIG. 15B a sectional view of the contact portion in a state in which optical fibers have been inserted;

FIG. 16 is a side view of the tip of the contact portion;

FIG. 17 is a sectional view of the tip of the contact portion;

FIG. 18 is a front view of the contact portion;

FIG. 19 is a perspective view of a mouthpiece as well as teeth enveloped by gums;

FIG. 20 is a perspective view illustrating the manner in which the mouthpiece is placed on teeth;

FIG. 21 is a plan view of the mouthpiece;

FIG. 22 is a plan view of the mouthpiece placed on teeth;

FIG. 23 is a sectional view taken along line XXIII-XXIII of FIG. 21;

FIG. 24 is a sectional view taken along line XXIV-XXIV of FIG. 21; and

FIG. 25 is a sectional view taken along line XXV-XXV of FIG. 21.

### BEST MODE FOR CARRYING OUT THE INVENTION

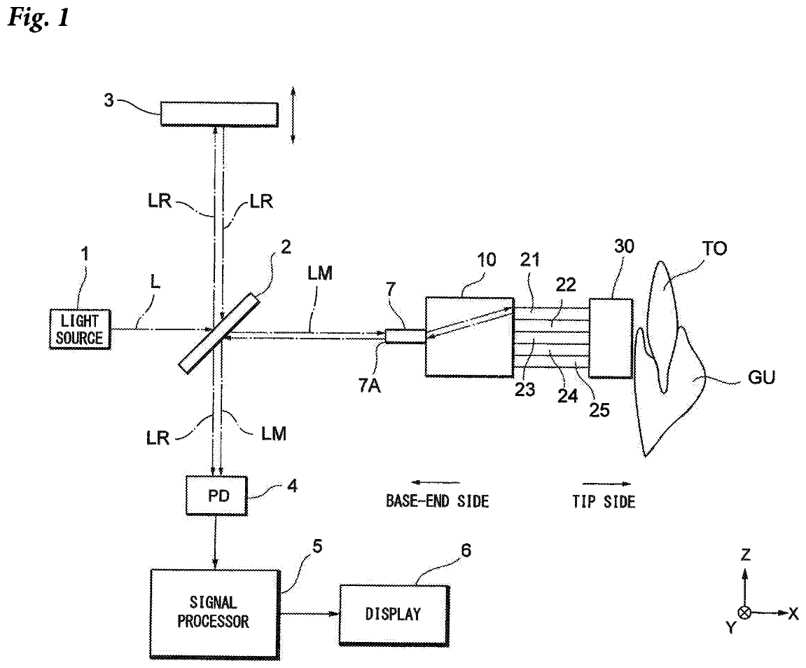

FIG. 1, which illustrates an embodiment of the present invention, is a block diagram showing the construction of a periodontal disease examination apparatus.

Low-interference light (low-coherence light) L is emitted from a light source 1 such as an SLD (Super Luminescent Diode). The low-interference light L is split into measuring light LM and reference light LR by a beam splitter 2 (one example of an optical divider). It will suffice if low-interference light L is emitted from the light source 1, and use may be made of another light source such as a gas laser, semiconductor laser or laser diode.

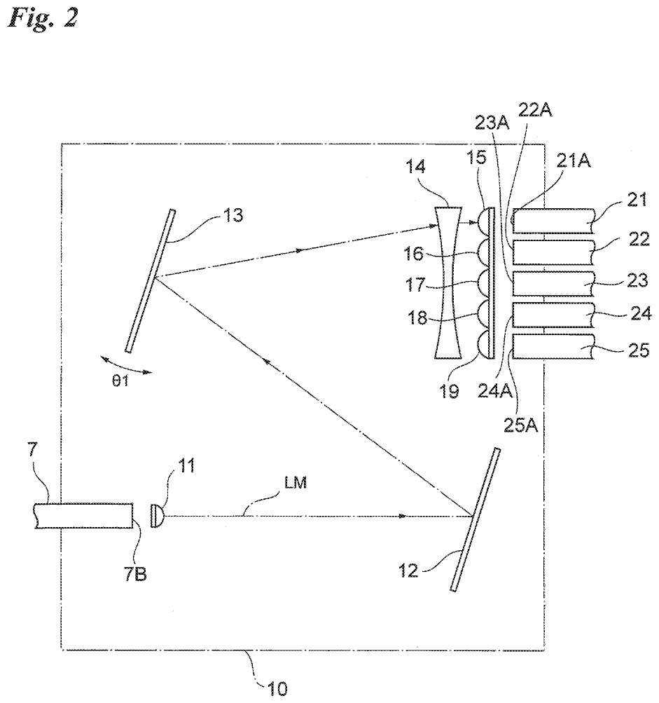

The measuring light LM split off by the beam splitter 2 impinges on a first optical fiber 7 (one example of a first optical waveguide) from a light-incident end face 7A of the first optical fiber 7. A light-emitting end face 7B (see FIG. 2, etc.) of the first optical fiber 7 is connected to a deflecting unit 10. Connected to the deflecting unit 10 are five (five is the number adopted for the sake of convenience but the number may be more or less than five) second optical fibers 21 to 25 (an example of a plurality of second optical waveguides) (the five second optical fibers 21 to 25 are an example of an optical waveguide). The measuring light LM emitted from the measuring-light-LM light-emitting end face 7B (see FIG. 2, etc.) of the first optical fiber 7 is deflected by the deflecting unit 10 so as to impinge successively on each of measuring-light-LM-incident end faces 21A to 25A (see FIG. 2, etc.) of the five optical fibers 21 to 25. The measuring light LM that has impinged on the second optical fibers 21 to 25 propagates through the second optical fibers 21 to 25, passes through an examination probe 30, is emitted from measuring-light-LM light-emitting end faces 21B to 25B of the second optical fibers 21 to 25 and irradiates a gum GU and a tooth TO which are to undergo measurement.

The measuring light LM that has irradiated the gum GU and tooth TO to undergo measurement is reflected from the gum GU and tooth TO. The measuring light LM reflected from the gum GU and tooth TO passes through the second optical fibers 21 to 25 and is guided to the first optical fiber 7 by the deflecting unit 10. The reflected measuring light LM is reflected in the beam splitter 2 and impinges on a photodiode 4 (one example of a photodetector).

Further, the reference light LR split off in the beam splitter 2 is reflected at a reference mirror 3 (reference surface) freely movable along the direction of propagation of the reference light LR and in the direction opposite thereto (along the positive and negative directions of the Z-axis in the embodiment shown in FIG. 1). The reflected reference light LR is transmitted through the beam splitter 2 and impinges upon the photodiode 4.

When, by moving the reference mirror 3, equality is established between a propagation distance, which is the sum total of propagation distance traveled until the measuring light LM irradiates the gum GU and tooth TO undergoing examination and propagation distance traveled until light reflected from the gum GU and tooth TO undergoing examination impinges upon the photodiode 4, and a propagation distance, which is the sum total of propagation distance traveled until the reference light LR irradiates the reference mirror 3 and light reflected from the reference mirror 3 impinges upon the photodiode 4, interference occurs between the measuring light LM and reference light LR and the photodiode 4 outputs an interference signal.

The interference signal output from the photodiode 4 is input to a signal processing circuit 5 (one example of periodontal pocket data generating device)(processor circuitry), and signals representing optical tomographic images of the gum GU and tooth TO (data regarding the depth of a periodontal pocket) are generated. By inputting the generated signals representing the optical tomographic images to a display unit 6, the optical tomographic images of the gum GU and tooth TO are displayed on the display screen of the display unit 6. Processing for extracting the contours of the optical tomographic images is executed in the signal processing circuit 5, whereby the depth of a periodontal pocket between the gum GU and tooth TO is calculated. The calculated depth of the periodontal pocket also is displayed on the display screen of the display unit 6. Although optical tomographic images are generated and the depth of the periodontal pocket is calculated from the generated optical tomographic images, an arrangement may be adopted in which, rather than generate optical tomographic images, numerical data representing the depth of the periodontal pocket (such numerical data also is considered to be data regarding the depth of the periodontal pocket) is calculated in the signal processing circuit 5 and the depth of the periodontal pocket is displayed on the display screen of the display unit 6.

In this embodiment, with regard to the optical fibers 7, 21 to 25 and the like, the portion in the direction in which the measuring light LM is emitted is taken as the tip side, and the portion in the direction in which the measuring light LM is reflected back is taken as the base-end side.

FIG. 2 illustrates the construction of the deflecting unit 10.

The first optical fiber 7 is connected to the deflecting unit 10 (one example of a deflecting mechanism), as mentioned above. A GRIN (gradient index) lens 11 is placed in front of the light-emitting end face 7B of the first optical fiber 7. (A GRIN lens is one example of a parallelizing element for outputting the incident light upon rendering it parallel. Another lens or optical element will also suffice as long as the incident light can be rendered parallel.) The measuring light LM rendered parallel by the GRIN lens 11 is reflected by a fixed mirror 12 (which does not rotate but which may be arranged to rotate) and guided to a deflecting mirror 13. The deflecting mirror 13 is rotatable through a predetermined angle and causes the incident light to be reflected at a deflection angle conforming to the angle of rotation. A MEMS (Micro-Electro-Mechanical Systems) mirror, for example, is adopted as the deflecting mirror 13. The measuring light LM reflected at the deflecting mirror 13 is rendered parallel by an f−θ lens 14 (one example of a parallelizing element for outputting the incident light upon rendering it parallel; may just as well be another parallelizing element), passes through any of condensing lenses 15 to 19 and impinges on any of the second optical fibers 21 to 25 from the light-incident end faces 21A to 25A of the second optical fibers 21 to 25. It should be noted that the meaning of the term “parallelizing” is not limited to making light perfectly parallel but is a concept that also includes making light approximately parallel. Further, in this embodiment, it is preferred that the parallelizing element render light slightly condensed rather than perfectly parallel. That is, it is preferred that the effects of attenuation of light and of diffusion when light is transmitted through a substance be reduced, and that the focal point of the light not be situated in close proximity to the parallelizing element.

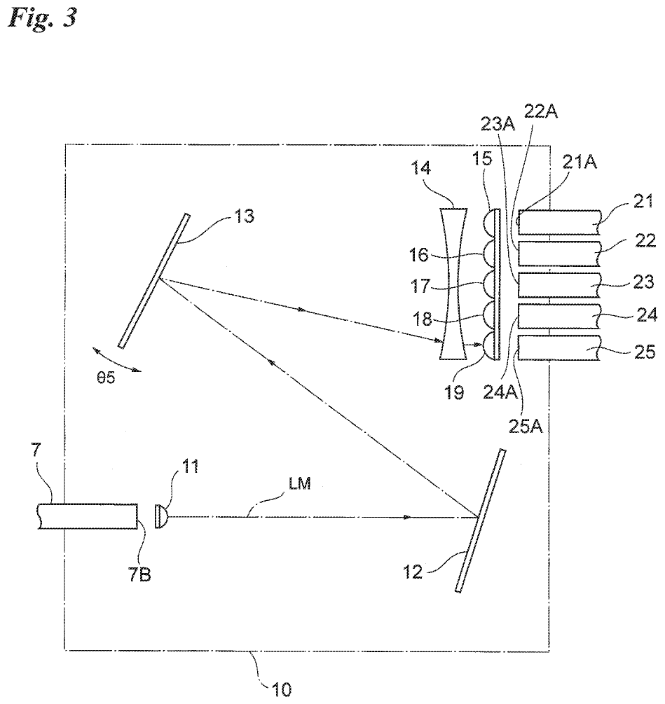

By controlling the angle of rotation of the deflecting mirror 13 using a control unit (not shown), the measuring light LM can be made to impinge on any of the second optical fibers 21 to 25. For example, by rotating the deflecting mirror 13 through an angle θ₁ from a predetermined angle, the measuring light LM will impinge on the second optical fiber 21 through the condensing lens 15, as illustrated in FIG. 2. Similarly, when the deflecting mirror 13 is rotated through an angle θ₂, θ₃ or θ₄ from a predetermined angle, the measuring light LM will impinge on the second optical fiber 22, 23 or 24 through the condensing lens 16, 17 or 18. When the deflecting mirror 13 is rotated through an angle θ₅ from a predetermined angle, the measuring light LM will impinge on the second optical fiber 25 through the condensing lens 19, as shown in FIG. 3.

As mentioned above, the measuring light LM emitted from the measuring-light-LM light-emitting end faces of the second optical fibers 21 to 25 is reflected at the gum GU and tooth TO and again impinges on the second optical fibers 21 to 25 from their light-emitting end faces that emitted the light. The measuring light LM that has again impinged on the second optical fibers 21 to 25 after being reflected at the gum GU and tooth TO is again incident upon the first optical fiber 7 via a path that is the reverse of the path on which the light is emitted from the first optical fiber 7 to the second optical fibers 21 to 25.

The control unit that controls the angle of rotation of the deflecting mirror 13 and this deflecting mirror are, respectively, one example of a first control mechanism for controlling the measuring light LM such that the measuring light LM that has been emitted from the first optical fiber 7 will impinge successively on the five second optical fibers 21 to 25, and one example of a deflecting mechanism for deflecting the measuring light LM that has been emitted from the first optical fiber 7 and guiding the measuring light to each of the five second optical fibers 21 to 25 constituting the optical fiber array.

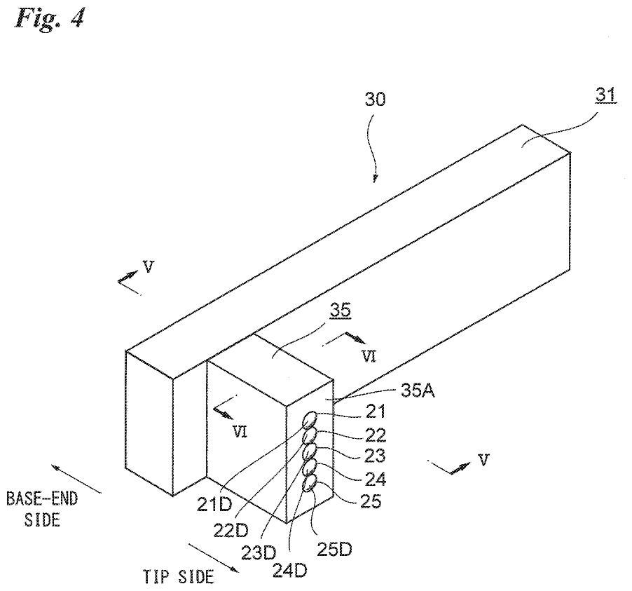

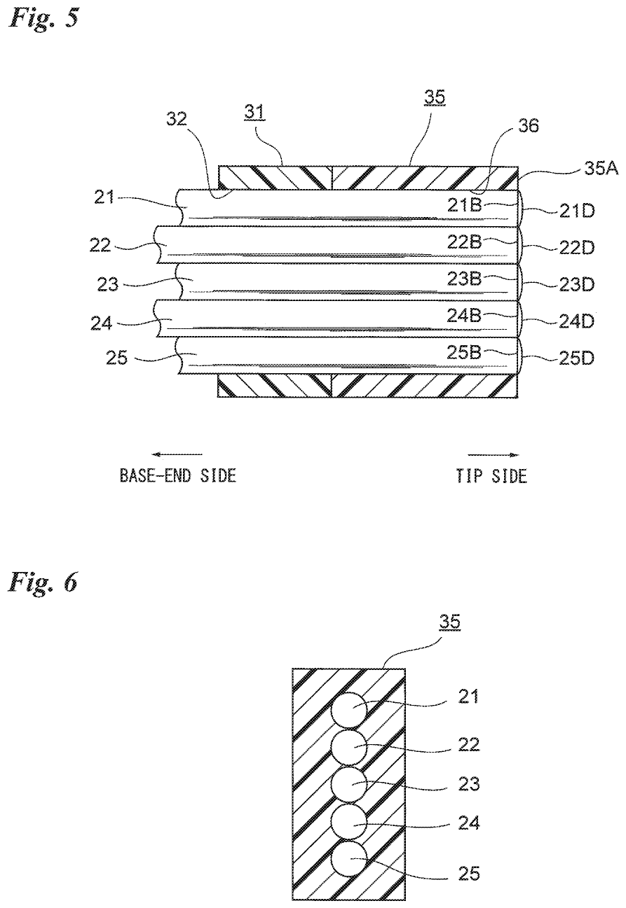

FIG. 4 is a perspective view of the examination probe 30, FIG. 5 a sectional view taken along line V-V of FIG. 4, and FIG. 6 a sectional view taken along line VI-VI of FIG. 4.

As illustrated in FIG. 4, the examination probe 30 includes a gripping portion 31 extending in one direction, and a contact portion 35 located at one end of the gripping portion 31 and extending from the gripping portion 31 in a direction perpendicular thereto.

As shown in FIG. 5, the gripping portion 31 is formed to have a space portion 32 from its base-end side toward its contact side. The contact portion 35 also is formed to have a space portion 36 from its base-end side toward its contact side. With reference also to FIG. 6, the five second optical fibers 21 to 25 arrayed in a single row pass through the interior of the space portions 32 and 36 from the base-end side to the tip side of the examination probe 30. The measuring-light-emitting end faces 21B to 25B of the five optical fibers 21 to 25 are exposed at a tip face 35A of the contact portion 35. Further, GRIN lenses 21D to 25D are secured to respective ones of the measuring-light-emitting end faces 21B to 25B of the five optical fibers 21 to 25. The GRIN lenses 21D to 25D, similar to the GRIN lens 11 with which the first optical fiber 7 is equipped, are one example of parallelizing elements for rendering parallel the measuring light LM emitted from the light-emitting end faces 21B to 25B; other lenses or optical elements will also suffice as long as the light can be rendered parallel. Further, the invention is not limited to a case where each of the GRIN lenses 21D to 25D (parallelizing elements) and the respective ones of the five optical fibers 21 to 25 are bodies separate from each other. By subjecting the tips of the five optical fibers 21 to 25 to machining such as grinding, parallelizing elements that function as the GRIN lenses 21D to 25D may be formed on the tips of the second optical fibers 21 to 25, thereby integrating each of the GRIN lenses 21D to 25D (parallelizing elements) and the respective ones of the five optical fibers 21 to 25. It should be noted that, irrespective of the case where the GRIN lenses 21D to 25D (parallelizing elements) and the respective ones of the five optical fibers 21 to 25 are separate from each other or the case where they are integrated, the GRIN lenses 21D to 25D (parallelizing elements) and the five optical fibers 21 to 25 are one example of second optical waveguides. Further, the diameter of each of the GRIN lenses 21D to 25D may be smaller than the diameter of each of the second optical fibers 21 to 25. That is, it will suffice if the diameter of each of the GRIN lenses 21D to 25D is sized such that the area where the measuring light LM is emitted from each of the light-emitting end faces 21B to 25B can be covered by the lens.

The gripping portion 31 is produced using a hard resin as the material and hardly expands or contracts. The contact portion 35 is made of a soft resin (polyurethane, for example), which is a flexible material, and the expansion/contraction rate thereof is higher than a predetermined threshold value (it expands and contracts comparatively easily). Of the outer peripheral surface of each of the five second optical fibers 21 to 25, the surface that contacts the space portion 32 formed in the inner wall of the gripping portion 31 (at the boundary surface between the gripping portion 31 and the space portion 32) simply passes through the space portion 32 without being affixed to the inner wall of the gripping portion 31. On the other hand, of the outer peripheral surface of each of the five second optical fibers 21 to 25, at least a portion of the surface that contacts the space portion 36 formed in the inner wall of the contact portion 35 is in contact with the inner wall of the gripping portion 31. The five second optical fibers 21 to 25 are not affixed to each other and, irrespective of whether there is mutual contact between them, are independent and free to move independently along the direction of the optical axis. For example, the outer peripheral surface of each of the five second optical fibers 21 to 25 has such high smoothness that, even if the outer peripheral surfaces of a plurality of the second optical fibers are in contact each other, each of the second optical fibers is capable of sliding. As shown in FIG. 5, therefore, even in a case where the five second optical fibers 21 to 25 are in contact with each other, each optical fiber is independent and free to move independently along the direction of the optical axis. Alternatively, the space portion 36 of the contact portion 35 may be formed such that the five second optical fibers 21 to 25 will not come into contact with each other at their outer peripheral surface. For example, the space portion 36 of the contact portion 35 may be a plurality of (five in this case) insertion holes, which are provided in the interior of the contact portion 35, through which the respective five second optical fibers 21 to 25 are capable of being inserted. Therefore, even in a case where the outer peripheral surface of each of the five second optical fibers 21 to 25 does not possess a high smoothness, each optical fiber will be independent and free to move independently along the direction of the optical axis. The contact portion 35 is produced from a soft resin, which is a flexible material. Therefore, in a case where a force toward the base-end side is applied to any of the second optical fibers among the second optical fibers 21 to 25, only the second optical fiber to which the force is applied will move toward the base-end side against a tip-side return force produced by the contact portion 35, and the other second optical fibers will be acted on by the tip-side return force produced by the contact portion 35. Accordingly, only the second optical fiber to which the force toward the base-end side is applied will move toward the base-end side. Thus, the optical fiber array (one example of an optical waveguide array) constituted by the five second optical fibers 21 to 25 is held by the contact portion 35 (one example of a holder), which comprises a flexible material, such that the optical fibers are independent and free to move along the direction of the optical axes of the respective second optical fibers 21 to 25 (if a certain second optical fiber moves along the direction of its optical axis, the other second optical fibers will not move together).

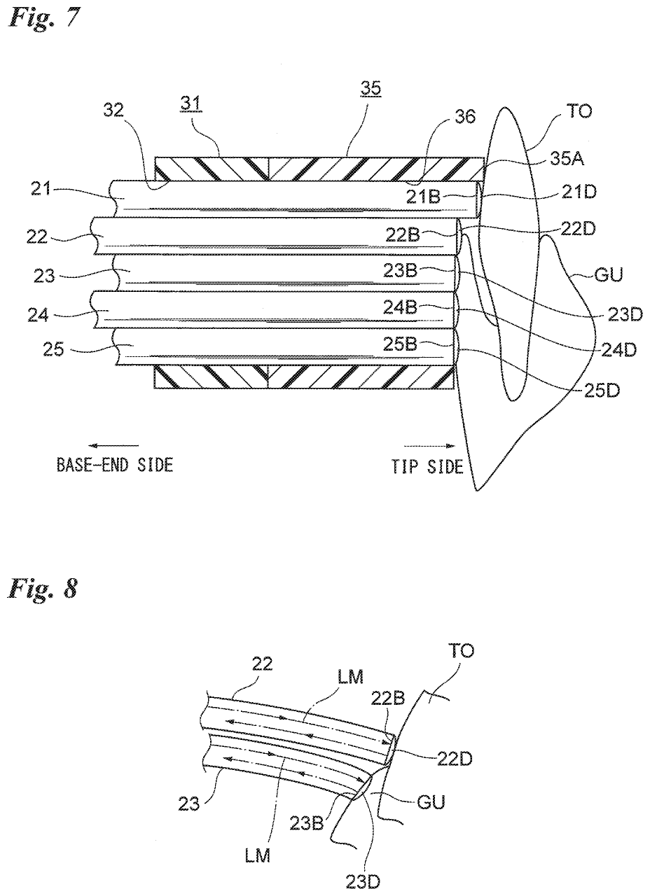

FIGS. 7 and 8 illustrate the manner in which the tip face 35A of the contact portion 35 of examination probe 30 is brought into contact with the gum GU and tooth TO to undergo examination. FIG. 8 illustrates a portion extracted from FIG. 7.

Generally, the surfaces of the gum GU and tooth TO are not planar but are complicated in shape. Owing to the fact that the contact portion 35 is formed of a flexible material, when the tip face 35A of the contact portion 35 is brought into contact with the gum GU and tooth TO, the contact portion 35 undergoes deformation such that the tip face 35A of the contact portion 35 follows the shape of the surfaces of the gum GU and tooth TO. At such time each of the light-emitting end faces 21B to 25B of the second optical fibers 21 to 25 is subjected to a force, which is directed toward the base-end side, in accordance with the shape of the surfaces of the gum GU and tooth TO. Here, since the second optical fibers 21 to 25 are freely movable independently along the direction of the optical axes of the respective second optical fibers 21 to 25, the light-emitting end faces 21B to 25B of the second optical fibers 21 to 25 come into close contact with the surfaces of the gum GU and tooth TO via the respective GRIN lenses 21D to 25D. For example, as shown in FIG. 8, although the surface of the gum GU protrudes from the surface of the tooth TO, the light-emitting face 22B of the second optical fiber 22 comes into close contact with the surface of the tooth TO via the GRIN lens 22D, and the light-emitting face 23B of the second optical fiber 23 comes into close contact with the surface of the gum GU via the GRIN lens 23D. Since the light-emitting end faces 21B to 25B of the second optical fibers 21 to 25 thus come into close contact with the surfaces of the gum GU and tooth TO via the respective GRIN lenses 21D to 25D, measuring light LM, which is reflected from the gum GU and tooth TO and, when a periodontal pocket exists, from the boundary surface of the tooth TO and periodontal pocket, can be made to impinge on the second optical fibers 21 to 25 without loss.

Further, since a portion of the outer peripheral surface of each of the second optical fibers 21 to 25 is affixed (secured) to the contact portion 35, the tips of the second optical waveguides, namely the light-emitting end faces 21B to 25B of the second optical fibers 21 to 25 or the GRIN lenses 21D to 25D provided on these end faces, are prevented from receding toward the base-end side further than the tip face 35A of the contact portion 35. If we suppose a case where the second optical fibers 21 to 25 should happen to recede toward the base-end side, the light-emitting end faces 21B to 25B of the second optical fibers 21 to 25 and the surfaces of the gum GU and tooth TO will not come into close contact via the grin lenses 21D to 25D even in a state in which the tip face 35A of the contact portion 35 is in contact with surfaces of the gum GU and tooth TO. As a result, in such case the amount of light reflected from the gum GU and tooth TO that would enter the second optical fibers 21 to 25 would decrease and loss would increase. Still, it is not necessarily required for the second optical fibers 21 to 25 to be fixed to the contact portion 35 (holder) in such a manner that the second optical fibers 21 to 25 will not recede toward the base-end side.

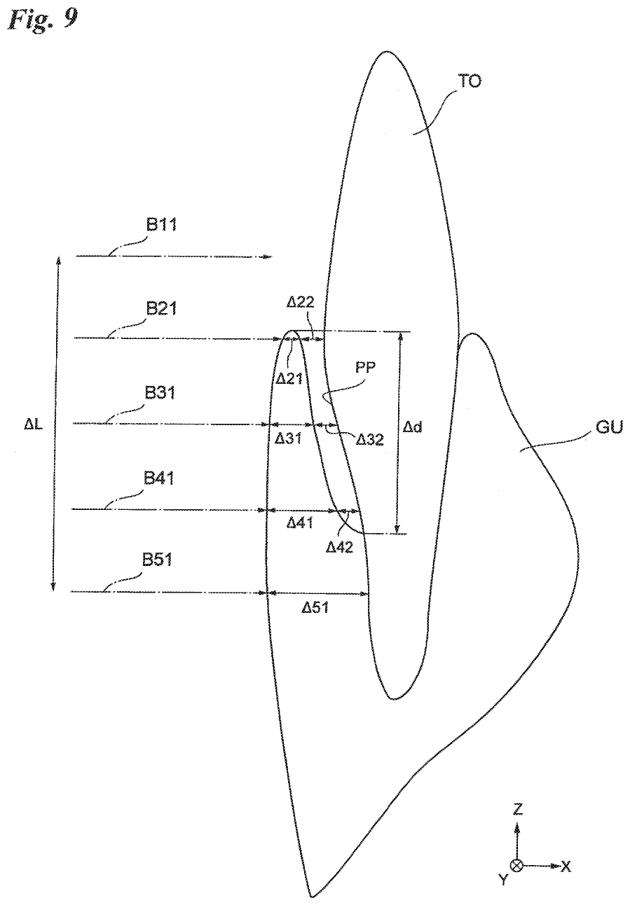

FIG. 9 illustrates the manner in which the gum GU and the tooth TO undergoing examination are irradiated with measuring light beams B11, B21, B31, B41 and B51. FIG. 9 is enlarged as compared with FIGS. 1 and 7. Illustration of the optical fibers 21 to 25 is omitted in FIG. 9.

The measuring light beam B11 is the measuring light LM that propagates through the second optical fiber 21. Similarly, the measuring light beams B21, B31, B41 and B51 are beams of the measuring light LM propagating through the second optical fibers 22, 23, 24 and 25, respectively.

In FIG. 9, the gum GU and tooth TO are as seen from the side. The left side in FIG. 9 corresponds to one of either the outside or the inside of the body, and the right side corresponds to the other one of either the outside or the inside of the body.

A periodontal pocket PP has formed between the gum GU and tooth TO. In the case of severe periodontal disease, the depth of the periodontal pocket PP is 6 mm or more. If deflection width ΔL of the measuring light beams B11 to B51 (deflection width of the measuring light beams B11 to B51 along the depth direction of the periodontal pocket PP) is 6 mm or more, therefore, whether the periodontal pocket PP exhibits sever periodontal disease can be determined. Accordingly, the number of the second optical fibers 21 to 25 and the diameter of each of the second optical fibers 21 to 25 are decided in such a manner that the deflection width ΔL of the measuring light beams B11 to B51 will be 6 mm or more. Thus, enough deflection width to measure the depth of a periodontal pocket in a single scan is preferred.

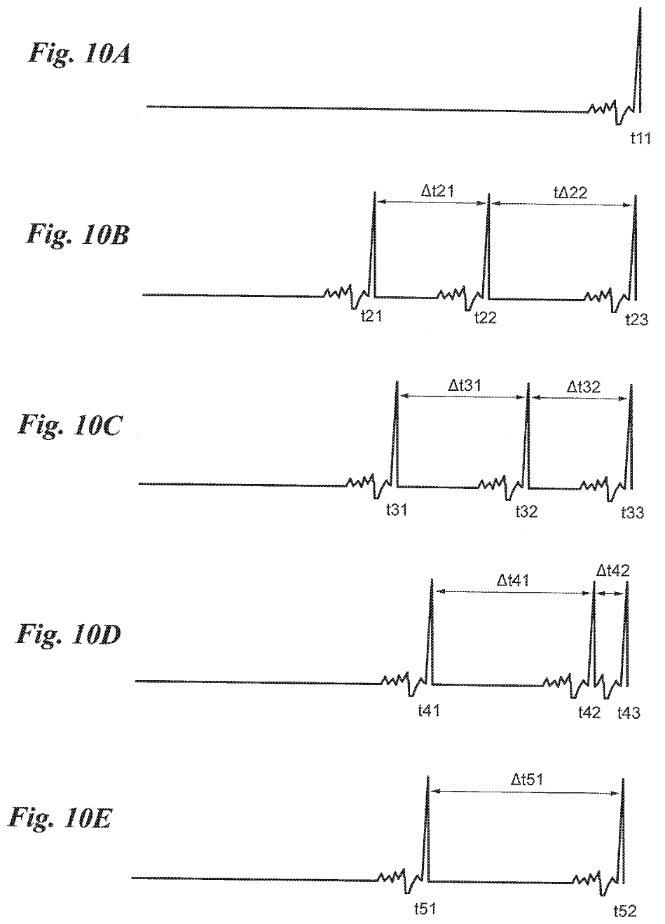

FIG. 10A to FIG. 10E are examples of interference signals.

FIG. 10A, FIG. 10B, FIG. 10C, FIG. 10D and FIG. 10E are examples of interference signals obtained based on the measuring light beams B11, B21, B31, B41 and B51, respectively.

The measuring light beam B11 directly irradiates the portion of the tooth TO where the gum GU is not present (see FIG. 9), and the intensity of the light reflected from the surface of the tooth TO rises. Based on the light reflected from the surface of the tooth TO, therefore, an interference signal is generated at time t11, as illustrated in FIG. 10A.

Since the measuring light beam B21 irradiates the upper end of the periodontal pocket PP (see FIG. 9), the intensity of the light reflected from the surface of the gum GU, from the boundary between the gum GU and the periodontal pocket PP, and from the surface of the gum GU, rises. As illustrated in FIG. 10B, therefore, interference signals are generated at times t21, t22 and t23, as shown in FIG. 10B, based on the light reflected from the surface of the gum GU, from the boundary between the gum GU and the periodontal pocket PP, and from the surface of the tooth TO, respectively. A time difference Δt21 from time t21 to time t22 indicates thickness 421 of the gum GU at the portion irradiated with the measuring light beam B21, and a time difference Δt22 from time t22 to time t23 indicates distance Δ22 across the gap (the distance across the space between the tooth TO and gum GU) of the periodontal pocket PP at the portion irradiated with measuring light beam B21.

Similarly, interference signals are generated at times t31, t32 and t33, as illustrated in FIG. 10C, based on the light reflected from the surface of the gum GU, from the boundary between the gum GU and the periodontal pocket PP, and from the surface of the tooth TO, respectively, owing to irradiation with the measuring light beam B31. A time difference Δt31 from time t31 to time t32 indicates thickness 431 of the gum GU at the portion irradiated with the measuring light beam B31, and a time difference Δt32 from time t32 to time t33 indicates distance 432 across the gap of the periodontal pocket PP at the portion irradiated with measuring light beam B31.

Similarly, interference signals are generated at times t41, t42 and t43, as illustrated in FIG. 10D, based on the light reflected from the surface of the gum GU, from the boundary between the gum GU and the periodontal pocket PP, and from the surface of the tooth TO, respectively, owing to irradiation with the measuring light beam B41. A time difference Δt41 from time t41 to time t42 indicates thickness 441 of the gum GU at the portion irradiated with the measuring light beam B41, and a time difference Δt42 from time t42 to time t43 indicates distance 442 across the gap of the periodontal pocket PP at the portion irradiated with measuring light beam B41.

No periodontal pocket PP has formed at the portion of the gum GU irradiated with the measuring light beam B51 (see FIG. 9). Based on the light reflected from the gum GU and from the surface of the tooth TO owing to irradiation with the measuring light beam B5, therefore, interference signals are generated at times t51 and t52, as illustrated in FIG. 10E. A time difference Δt51 from time t51 to time t52 indicates thickness 451 of the gum GU at the portion irradiated with the measuring light beam B51.

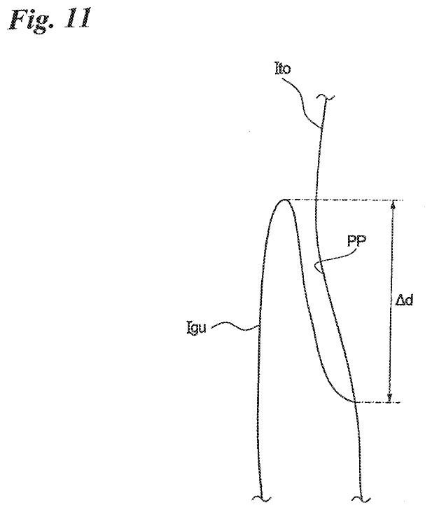

Optical tomographic images of the gum GU and tooth TO shown in FIG. 11 are generated by plotting the peak values of the interference signals of FIG. 10A to FIG. 10E.

FIG. 11 is an example of an optical tomographic image Igu of the gum GU and an optical tomographic image Ito of the tooth TO.

The optical tomographic image Igu of the gum GU and the optical tomographic image Ito of the tooth TO are displayed on the display screen of the display unit 6. By subjecting the optical tomographic image Igu of the gum GU and the optical tomographic image Ito of the tooth TO to contour extraction in the signal processing circuit 5, the depth Δd of the periodontal pocket PP is calculated in the signal processing circuit 5.

In above-described embodiment, the depth Δd of the periodontal pocket PP is calculated by generating the optical tomographic images Igu and Ito of the gum GU and tooth TO and extracting the contours of the generated optical tomographic images Igu and Ito. However, the depth Δd of the periodontal pocket PP may be calculated by computation without generating the optical tomographic images Igu and Ito (although the optical tomographic images Igu and Ito may just as well be generated).

In the embodiment set forth above, it is assumed that the deflection width from the measuring light beam B11 to B51 is enough to enable measurement of the depth Δd of the periodontal pocket in a single scan even in case of severe periodontal disease. However, in instances where there is not enough deflection width to enable measurement of the depth Δd of the periodontal pocket in a single scan, an arrangement may be adopted in which, by performing measurement multiple times at positions that differ in height (at least at two locations), data regarding the depth Δd of the periodontal pocket will be generated in the signal processing circuit (a periodontal pocket data generating device) 5 based upon interference signals output from the photodiode 4.

For example, assume that the examination probe 30 can emit measuring light having a deflection width corresponding to the range from measuring light beam B11 to B31 (equal to the range from B31 to B51), which is illustrated in FIG. 9, by a single scan (measurement). First, assume that a first scan (measurement) by the examination probe 30 is carried out, at positions at which measuring light is capable of being emitted, over a range corresponding to the measuring light beams B11 to B31 illustrated in FIG. 9. In this case, optical tomographic images Igu and Ito of the upper half of gum GU and tooth TO shown in FIG. 9 are obtained from interference signals based on measuring light emitted over the range from measuring light beam B11 to B31, shown in FIG. 9, in the first scan. Next, the examination probe 30 is moved downward. Assume that, owing to a second scan performed at the position of the probe after such movement, measuring light from the examination probe 30 is emitted over the range from measuring light beam B31 to B51 shown in FIG. 9. In this case, optical tomographic images Igu and Ito of the lower half of gum GU and tooth TO shown in FIG. 9 are obtained from interference signals based on measuring light emitted over the range from measuring light beam B31 to B51, shown in FIG. 9, in the second scan. By subjecting the two optical tomographic images, which have been obtained by measurement performed at the positions of two points of different height, to combining processing in the signal processing circuit 5, the optical tomographic images of the gum GU and tooth TO shown in FIG. 9 are obtained. Needless to say, the optical tomographic images Igu and Ito of the upper half of gum GU and tooth TO and the optical tomographic images Igu and Ito of the lower half of gum GU and tooth TO are combined so as to be superimposed with regard to the overlapping portions thereof, and the connectivity of the optical tomographic images in the vertical direction is assured so as to obtain optical tomographic images that will be identical to the optical tomographic images Igu and Ito that would be obtained by a single scan.

In the foregoing embodiment, the second optical fibers 21 to 25 are arrayed in a single row but they may be arrayed in two rows or more. In such case the deflecting mirror 13 would not deflect the measuring light LM only in a one dimensional direction but would be arranged so as to be able to deflect the measuring light LM in the directions of two dimensions and adapted so as to guide the measuring light to the optical fibers included in each row. Further, the five optical fibers 21 to 25 need not necessarily be arrayed on a straight line and may be arranged along a curving line.

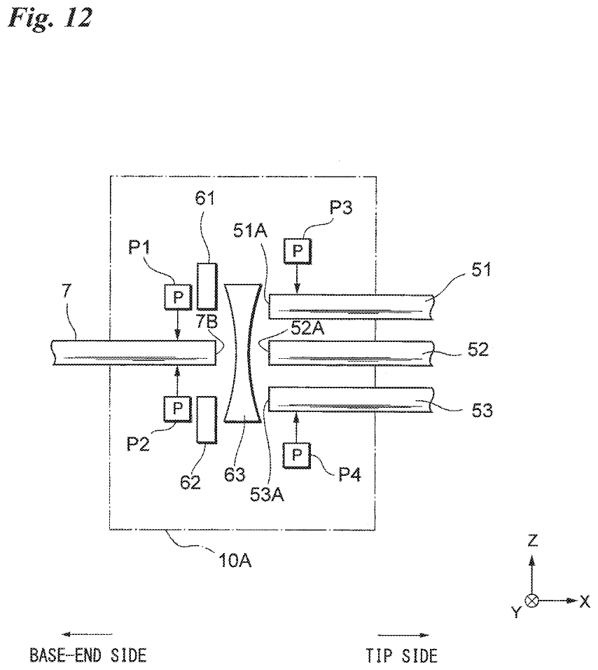

FIG. 12 illustrates another example of the deflecting unit.

A deflecting unit 10A shown in FIG. 12 utilizes piezoelectric elements. In FIG. 12, the direction to the right is the X-axis direction, the direction into the drawing is the Y-axis direction and the upward direction is the Z-axis direction. The X-axis direction is in the direction of the tip side and the negative direction along the X-axis is in the direction of the base-end side.

Piezoelectric elements P1 and P2 are fixed to the tip of the first optical fiber 7. Stoppers 61 and 62 are provided closer to the tip than are the piezoelectric elements P1 and P2 and in such a manner that the first optical fiber 7 is interposed between them. The stoppers 61 and 62 limit movement of the first optical fiber 7 in the up-and-down direction (the Z-axis direction and negative direction along the Z-axis). An f−θ lens 63 is provided in front of the first optical fiber 7. A second optical fiber 52 is provided at a position where it faces the first optical fiber 7 with the f−θ lens 63 interposed therebetween. A second optical fiber 51 is provided above the second optical fiber 52, and a second optical fiber 53 is provided below the second optical fiber 52. A piezoelectric element P3 is fixed to the base end of the second optical fiber 51, and a piezoelectric element P4 is fixed to the base end of the second optical fiber 53.

The tip of the first optical fiber 7 is moved downward by the piezoelectric element P1 fixed to the first optical fiber 7, and the tip of the first optical fiber 7 is moved upward by the piezoelectric element P2 fixed to the first optical fiber 7. The position of the measuring-light-LM-emitting end face of the first optical fiber 7 is adjusted by the piezoelectric elements P1 and P2.

Further, among the plurality of second optical fibers 51, 52 and 53, the base end of the second optical fiber 51 is moved downward by the piezoelectric element P3 fixed to the base end of the second optical fiber 51, and the base end of the second optical fiber 53 is moved upward by the piezoelectric element P4 fixed to the base end of the second optical fiber 53. The positions of the measuring-light-LM-incident end faces of the second optical fibers 51 and 53 are adjusted by the piezoelectric elements P3 and P4.

The second optical fibers 51, 52 and 53 are held by the contact portion 35 (holder) of the examination probe 30 shown in FIG. 4. Needless to say, the second optical fibers 51, 52 and 53 are held spaced apart by the contact portion 35 such that the base end of the second optical fiber 51 can move downward and the base end of the second optical fiber 53 can move upward.

By virtue of the fact that tip of the first optical fiber 7 is moved upward by the piezoelectric element P2 and the base end of the second optical fiber 51 is moved downward by the piezoelectric element P3, the measuring-light-LM emitting end face 7B of the first optical fiber 7 and the measuring-light-LM incident end face 51A of the second optical fiber 51 come to oppose each other. In a case where none of the piezoelectric elements P1 to P4 operate, the measuring-light-LM emitting end face 7B of the first optical fiber 7 and the measuring-light-LM incident end face 52A of the second optical fiber 52 will oppose each other. Owing to the fact that tip of the first optical fiber 7 is moved downward by the piezoelectric element P1 and the base end of the second optical fiber 53 is moved upward by the piezoelectric element P4, the measuring-light-LM emitting end face 7B of the first optical fiber 7 and the measuring-light-LM incident end face 54A of the second optical fiber 54 come to oppose each other. The voltages applied to the piezoelectric elements P1 to P4 are adjusted by a voltage circuit and voltage control circuit (neither of which is shown) such that measuring-light-LM emitting end face 7B of the first optical fiber 7 will successively oppose the measuring-light-LM incident end faces 51A, 52A and 53A of the plurality of second optical fibers 51, 52 and 53. Since the f−θ lens 63 (one example of a parallelizing element for rendering the measuring light parallel) is provided in front of second optical fibers 51 to 53, the rate of incidence of the measuring light LM on the second optical fibers 51 to 53 rises.

The piezoelectric elements P1 to P4, the voltage circuit and the voltage control circuit are a first control mechanism for controlling the first optical fiber 7 and at least one among the second optical fibers 51, 52 and 53 such that the measuring light LM emitted from the first optical fiber 7 will successively impinge on the plurality of second optical fibers 51, 52 and 53 constituting the optical fiber array, as well as a first adjusting mechanism for adjusting at least one of: the position of the measuring-light-LM emitting end face 7B of the first optical fiber 7, and positions of the measuring-light-LM incident end faces 51A, 52A and 53A of the plurality of second optical fibers 51, 52 and 53, such that the measuring-light-LM emitting end face 7B of the first optical fiber 7 (first optical waveguide) will successively oppose the measuring-light-LM incident end faces 51A, 52A and 53A of the second optical fibers 51, 52 and 53 (second optical waveguides) constituting the optical fiber array (optical waveguide array).

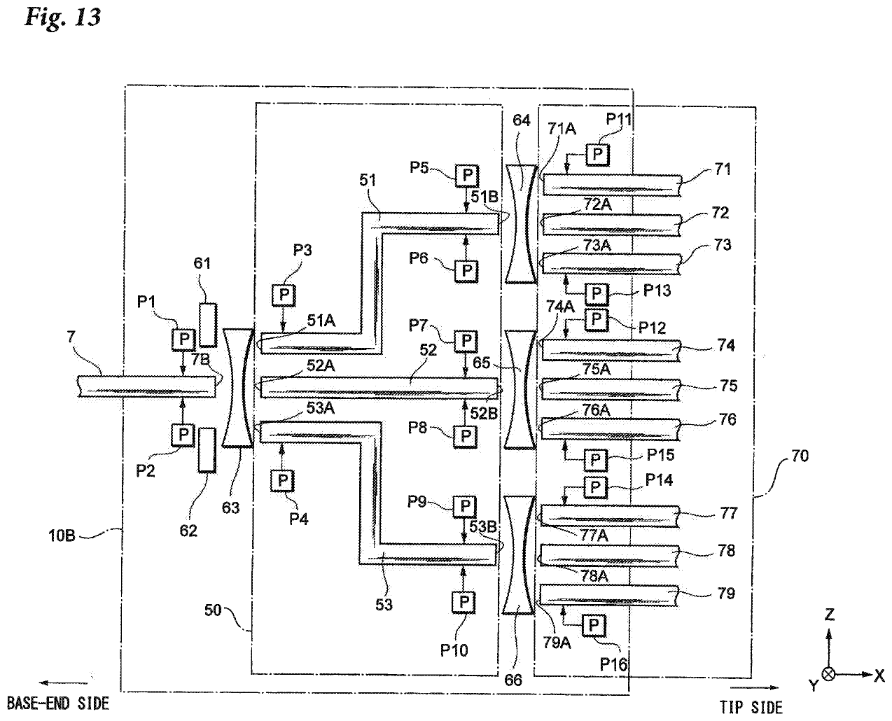

FIG. 13 is another example of the deflecting unit.

A deflecting unit 10B shown in FIG. 13 also utilizes piezoelectric elements. In FIG. 13 as well, the direction to the right is the X-axis direction, the direction into the drawing is the Y-axis direction and the upward direction is the Z-axis direction. The X-axis direction is in the direction of the tip side and the negative direction along the X-axis is in the direction of the base-end side. Components in FIG. 13 identical with those shown in FIG. 12 are designated by like symbols and a description thereof is omitted.

A second optical fiber array 50 included in the deflecting unit 10B includes the optical fibers 51, 52 and 53 as well as optical fibers 71 to 79. The optical fibers 51, 52 and 53 are a first optical fiber array 50, and the optical fibers 71 to 79 are a second optical fiber array 70. The optical fibers 51, 52 and 53 included in the first optical fiber array 50 are arrayed in at least one row, the measuring light LM emitted from the light-emitting end face 7B of the first optical fiber 7 enters from the measuring-light-LM-incident end faces 51A, 52A and 53A and is emitted from the measuring-light-LM-emitting faces 51B, 52B and 53B. The optical fibers 71 to 79 included in the second optical fiber array 70 are larger in number than the optical fibers 51, 52 and 53 included in the first optical fiber array 50, the measuring light LM emitted from the light-emitting faces 51B, 52B and 53B of the optical fibers 51, 52 and 53 included in the first optical fiber array 50 enters from measuring-light-LM incident end faces 71A to 79A and is emitted from measuring-light-LM emitting end faces 71B to 79B. The optical fibers 71 to 79 included in the second optical fiber array 70 are held by the contact portion 35 (holder) of the examination probe 30, as shown in FIG. 4.

In the first optical fiber array 50, a piezoelectric element P6 for moving the tip of the second optical fiber 51 upward and a piezoelectric element P5 for moving the tip of the second optical fiber 51 downward are fixed to the tip of the second optical fiber 51. Further, a piezoelectric element P8 for moving the tip of the second optical fiber 52 upward and a piezoelectric element P7 for moving the tip of the second optical fiber 52 downward are fixed to the tip of the second optical fiber 52. Furthermore, a piezoelectric element P10 for moving the tip of the second optical fiber 53 upward and a piezoelectric element P9 for moving the tip of the second optical fiber 53 downward are fixed to the tip of the second optical fiber 53.

In the second optical fiber array 70, a piezoelectric element P11 for moving the base end of the second optical fiber 71 downward is fixed to the base end of the second optical fiber 71, and a piezoelectric element P12 for moving the base end of the second optical fiber 72 upward is fixed to the base end of the second optical fiber 73. Further, a piezoelectric element P13 for moving the base end of the second optical fiber 74 downward is fixed to the base end of the second optical fiber 74, and a piezoelectric element P14 for moving the base end of the second optical fiber 76 upward is fixed to the base end of the second optical fiber 76. Furthermore, a piezoelectric element P15 for moving the base end of the second optical fiber 77 downward is fixed to the base end of the second optical fiber 77, and a piezoelectric element P16 for moving the base end of the second optical fiber 79 upward is fixed to the base end of the second optical fiber 79.

An f−θ lens 64 (another optical element will also suffice as long as it can make light parallel) for rendering parallel the measuring light LM emitted from the second optical fiber 51 is placed in front of the measuring-light-LM-incident end faces 71A, 72A and 73A (between the second optical fibers 71, 72 and 73 and the second optical fiber 51) of the second optical fibers 71, 72 and 73 in the second optical fiber array 70. Further, an f−θ lens 65 (another optical element will also suffice as long as it can make light parallel) for rendering parallel the measuring light LM emitted from the second optical fiber 52 is placed in front of the measuring-light-LM-incident end faces 74A, 75A and 76A (between the second optical fibers 74, 75 and 76 and the second optical fiber 52) of the second optical fibers 74, 75 and 76. Furthermore, an f−θ lens 66 (another optical element will also suffice as long as it can make light parallel) for rendering parallel the measuring light LM emitted from the second optical fiber 53 is placed in front of the measuring-light-LM-incident end faces 77A, 78A and 79A (between the second optical fibers 77, 78 and 79 and the second optical fiber 53) of the second optical fibers 77, 78 and 79.

The deflecting unit 10B includes the f−θ lens 63 provided between the first optical fiber 7 and the first optical fiber array 50 as well as the three f−θ lenses 64, 65 and 66 provided between the first optical fiber array 50 and the second optical fiber array 70. Although providing all these f−θ lenses 63, 64, 65 and 66 is preferred, it may be so arranged that, out of these f−θ lenses 63, 64, 65 and 66, at least one f−θ lens (parallelizing element) is provided; f−θ lenses need not necessarily be provided.

When the tip of the first optical fiber 7 is moved upward by the piezoelectric element P2 and the base end of the second optical fiber 51 is moved downward by the piezoelectric element P3, the measuring-light-LM emitting end face 7B of the first optical fiber 7 and the measuring-light-LM-incident end face 51A of the second optical fiber 51 come to oppose each other. The measuring light LM incident on the first optical fiber 7 impinges on the second optical fiber 51. When, in this state, the tip of the second optical fiber 51 is moved upward by the piezoelectric element P6 and the base end of the second optical fiber 71 is moved downward by the piezoelectric element P11, the measuring-light-LM emitting end face 51B of the second optical fiber 51 and the measuring-light-LM-incident end face 71A of the second optical fiber 71 come to oppose each other. The measuring light LM emitted from the second optical fiber 51, therefore, impinges on the second optical fiber 71. Further, when the tip of the second optical fiber 51 is not moved, the measuring-light-LM emitting end face 51B of the second optical fiber 51 and the measuring-light-LM-incident end face 72A of the second optical fiber 72 oppose each other. The measuring light LM emitted from the second optical fiber 51, therefore, impinges on the second optical fiber 72. Furthermore, when the tip of the second optical fiber 51 is moved downward by the piezoelectric element P5 and the base end of the second optical fiber 73 is moved upward by the piezoelectric element P12, the measuring-light-LM emitting end face 51B of the second optical fiber 51 and the measuring-light-LM-incident end face 73A of the second optical fiber 73 come to oppose each other. The measuring light LM emitted from the second optical fiber 51, therefore, impinges on the second optical fiber 73.

When the tip of the first optical fiber 7 is not moved up or down, the measuring-light-LM emitting end face 7B of the first optical fiber 7 and the measuring-light-LM-incident end face 52A of the second optical fiber 52 oppose each other. The measuring light LM incident on the first optical fiber 7 impinges on the second optical fiber 52. When, in this state, the tip of the second optical fiber 52 is moved upward by the piezoelectric element P8 and the base end of the second optical fiber 74 is moved downward by the piezoelectric element P13, the measuring-light-LM emitting end face 52B of the second optical fiber 52 and the measuring-light-LM-incident end face 74A of the second optical fiber 74 come to oppose each other. The measuring light LM emitted from the second optical fiber 52, therefore, impinges on the second optical fiber 74. Further, when the tip of the second optical fiber 52 is not moved, the measuring-light-LM emitting end face 52B of the second optical fiber 52 and the measuring-light-LM-incident end face 75A of the second optical fiber 75 oppose each other. The measuring light LM emitted from the second optical fiber 52, therefore, impinges on the second optical fiber 75. Furthermore, when the tip of the second optical fiber 52 is moved downward by the piezoelectric element P7 and the base end of the second optical fiber 76 is moved upward by the piezoelectric element P14, the measuring-light-LM emitting end face 52B of the second optical fiber 52 and the measuring-light-LM-incident end face 76A of the second optical fiber 76 come to oppose each other. The measuring light LM emitted from the second optical fiber 52, therefore, impinges on the second optical fiber 76.

When the tip of the first optical fiber 7 is moved downward by the piezoelectric element P1 and the base end of the second optical fiber 53 is moved upward by the piezoelectric element P4, the measuring-light-LM emitting end face 7B of the first optical fiber 7 and the measuring-light-LM-incident end face 53A of the second optical fiber 53 come to oppose each other. The measuring light LM incident on the first optical fiber 7 impinges on the second optical fiber 53. When, in this state, the tip of the second optical fiber 53 is moved upward by the piezoelectric element P10 and the base end of the second optical fiber 77 is moved downward by the piezoelectric element P15, the measuring-light-LM emitting end face 53B of the second optical fiber 53 and the measuring-light-LM-incident end face 77A of the second optical fiber 77 come to oppose each other. The measuring light LM emitted from the second optical fiber 53, therefore, impinges on the second optical fiber 77. Further, when the tip of the second optical fiber 53 is not moved, the measuring-light-LM emitting end face 53B of the second optical fiber 53 and the measuring-light-LM-incident end face 78A of the second optical fiber 78 oppose each other. The measuring light LM emitted from the second optical fiber 53, therefore, impinges on the second optical fiber 78. Furthermore, when the tip of the second optical fiber 53 is moved downward by the piezoelectric element P9 and the base end of the second optical fiber 77 is moved upward by the piezoelectric element P16, the measuring-light-LM emitting end face 53B of the second optical fiber 53 and the measuring-light-LM-incident end face 77A of the second optical fiber 77 come to oppose each other. The measuring light LM emitted from the second optical fiber 53, therefore, impinges on the second optical fiber 77.

Thus, by utilizing the piezoelectric elements P1 to P16, the measuring light LM emitted from the first optical fiber 7 can be made to propagate successively from the second optical fibers 71 to 79. Needless to say, the piezoelectric elements P1 to P16 are driven by voltages applied from a voltage circuit (not shown), and voltages for implementing the operation described above are applied to the corresponding piezoelectric elements by a voltage control circuit (not shown) that controls the voltage circuit. The piezoelectric elements P1 to P16, the voltage circuit and the voltage control circuit correspond to a second control mechanism for controlling at least one of: the optical fibers 51, 52 and 53 included in the first optical fiber array 50, and the optical fibers 71 to 79 included in the second optical fiber array 70, in such a manner that measuring light emitted from the optical fibers 51, 52 and 53 included in the first optical fiber array 50 impinges successively on the plurality of optical fibers 71 to 79 included in the second optical fiber array 70.

In the foregoing embodiment, the tip side of the first optical fiber 7 and both the base-end side of optical fiber 51 and base-end side of optical fiber 53 included in the first optical fiber array 50 are moved in order to make the measuring light LM emitted from the first optical fiber 7 impinge on optical fiber 51, 52 or 53. However, it may be so arranged that either the tip side of the first optical fiber 7 or the base-end side of the optical fiber 51 (or the base-end side of the optical fiber 53) included in the first optical fiber array 50 is moved. Similarly, it may be so arranged that either the tip of the optical fiber 51 included in the first optical fiber array 50 or the base-end side of the optical fiber 71 (or the base-end side of the optical fiber 73) included in the second optical fiber array 70 is moved. Further, it may be so arranged that either the tip of the optical fiber 52 included in the first optical fiber array 50 or the base-end side of the optical fiber 74 (or the base-end side of the optical fiber 76) included in the second optical fiber array 70 is moved, and it may be so arranged that either the tip of the optical fiber 53 included in the first optical fiber array 50 or the base-end side of the optical fiber 77 (or the base-end side of the optical fiber 79) included in the second optical fiber array 70 is moved.

Further, it may be so arranged that the measuring light LM emitted from the optical fibers 71 to 79 included in the second optical fiber array 70 is made to impinge on even more optical fibers.

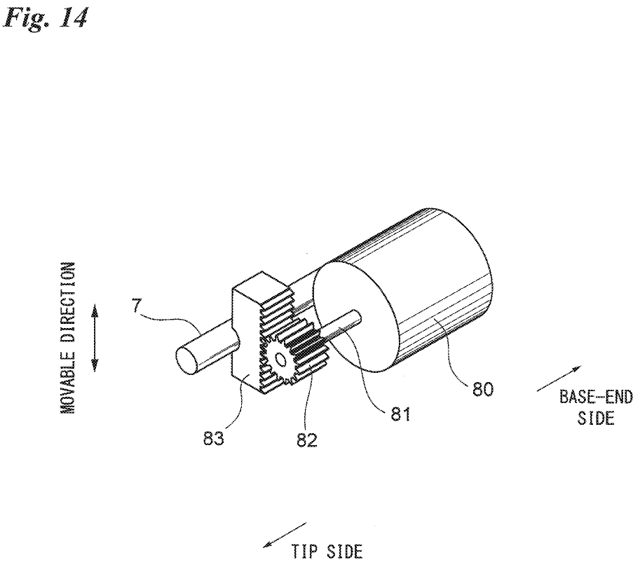

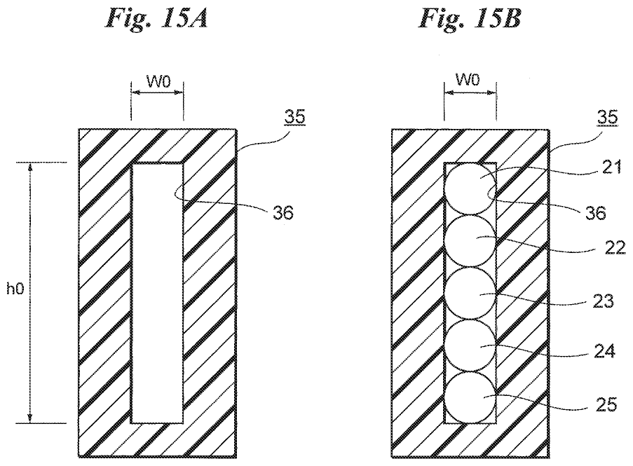

FIG. 14 is a perspective view of a stepping motor.

A rack 83 extending in the vertical direction is fixed to the side face of the first optical fiber 7 on the tip-side thereof. A pinion 82 fixed to shaft 81 of a stepping motor 80 is in mesh with the rack 83. Owing to rotation of the shaft 81 of stepping motor 80, the tip of the first optical fiber 7 moves upward or downward in accordance with the direction of rotation.

By fixing such a stepping motor 80, instead of the piezoelectric elements P1 to P4 shown in FIG. 12, to the tip of the first optical fiber 7 and to the base ends of the optical fibers 51, 52 and 53, the measuring light LM emitted from the first optical fiber 7 can be made to propagate to the optical fibers 51, 52 and 53. Similarly, by fixing such a stepping motor 80, instead of the piezoelectric elements P1 to P16 shown in FIG. 13, to the tip of the first optical fiber 7, the base end and the tip of the optical fiber 51, the tip of the optical fiber 52, the base end and tip of the optical fiber 53 and to the optical fibers 71, 73, 74, 76, 77 and 79, the measuring light LM emitted from the first optical fiber 7 can be made to propagate to the optical fibers 51, 52 and 53, as mentioned above, and the measuring light LM emitted from the first optical fiber 7 can be made to propagate to the optical fibers 71 to 79.

Figures from FIG. 15A and FIG. 15B to FIG. 18 are for describing a method of manufacturing the contact portion 35 that holds the second optical fibers 21 to 25.

FIG. 15A, which illustrates a state in which the second optical fibers 21 to 25 have not been inserted into the contact portion 35, corresponds to a sectional view taken along line VI-VI of FIG. 4.

From its base-end side to its tip side, the contact portion 35 is formed to have the space portion 36 through which pass the second optical fibers 21 to 25 arrayed in a single row. The space portion 36 has a width w0 substantially equal to the diameter of the second optical fibers 21 to 25 and a height h0 substantially equal to length in this case where the second optical fibers 21 to 25 are arrayed in a single row.

FIG. 15B, which illustrates a state in which the second optical fibers 21 to 25 have been inserted into the space portion 36, corresponds to a sectional view taken along line VI-VI of FIG. 4.

The second optical fibers 21 to 25 used have a diameter the same as the width w0 of the space portion 36 (though the diameter may be smaller or larger than the width w0). When the second optical fibers 21 to 25 are inserted into the space portion 36, the outer peripheral surface of each of the second optical fibers 21 to 25 and the inner wall of the contact portion 35, which inner wall forms the space portion 36, come into contact.

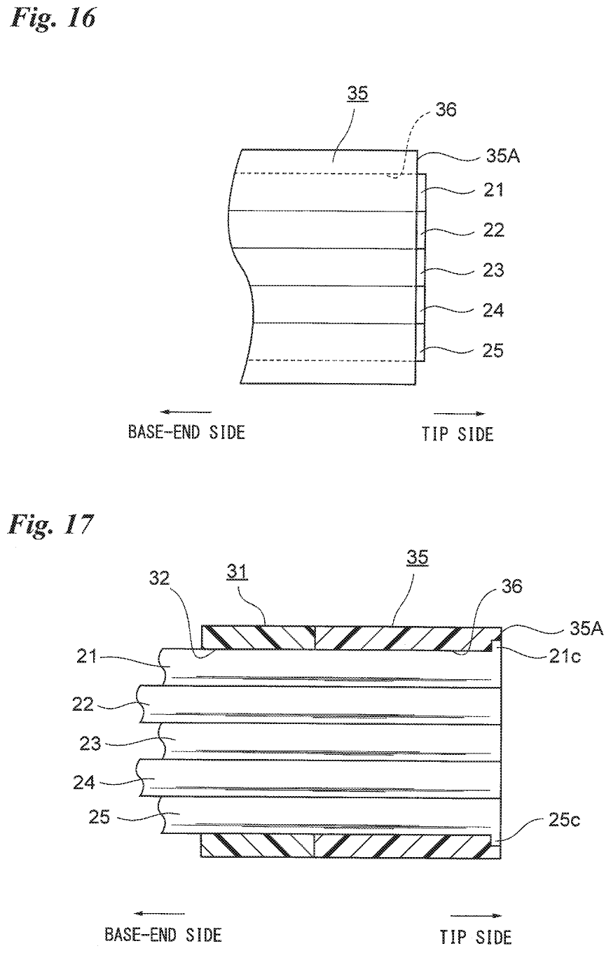

FIG. 16 is a side view illustrating the tip of the contact portion 35, and FIG. 17 is a sectional view showing the tip of the contact portion 35, this view corresponding to a sectional view taken along line V-V of FIG. 4.

As illustrated in FIG. 16, the second optical fibers 21 to 25 are inserted into the space portion 36 from the base-end side so as to protrude slightly from the tip face 35A of the contact portion 35. Thermal tightening is subsequently carried out so as to flatten out the tip faces of the second optical fibers 21 to 25, whereupon the tip faces of the second optical fibers 21 to 25 are flattened and become flush with the tip face 35A of the contact portion 35, as illustrated in FIG. 17. Since the tip faces of the second optical fibers 21 to 25 are flattened out, the diameter of the tip of each of the second optical fibers 21 to 25 is made larger than the diameter of the portion of each of the second optical fibers 21 to 25 other than the tip thereof. Thereafter, parallelizing elements such as the GRIN lenses 21D to 25D may be formed on the tips of the second optical fibers 21 to 25 by grinding, or the GRIN lenses 21D to 25D may be thermally bonded to the tip faces 21B to 25B of the second optical fibers 21 to 25. It should be noted that it will suffice if the diameter of the tip of each of the second optical fibers 21 to 25 is made larger than the diameter of the portion of each of the second optical fibers 21 to 25 other than the tip thereof. For example, in a case where the GRIN lenses 21D to 25D have been formed on the respective tip faces 21B to 25B of the second optical fibers 21 to 25, it will suffice if the diameter of each of the GRIN lenses 21D to 25D is less than the diameter of the portion of each of the second optical fibers 21 to 25 other than the tip thereof, as mentioned above.

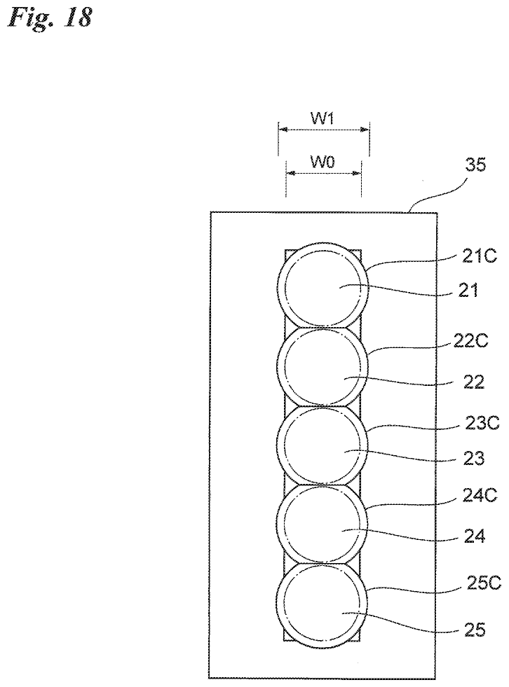

FIG. 18 is a front view of the contact portion 35 in a state in which the tip faces of the second optical fibers 21 to 25 have been flattened out.

The diameter (width) w1 of the tip of each of the second optical fibers 21 to 25 is larger than the width w0 of the space portion 36. Owing to thermal tightening of the tip faces of the second optical fibers 21 to 25, flanges 21C, 22C, 23C, 24C and 25C that spread in the radial direction are formed on the tips of these optical fibers. The flanges 21C, 22C, 23C, 24C and 25C penetrate into the contact portion 35, as shown in FIG. 17, owing to softening of the tip of the contact portion 35 due to thermal tightening.

As a result, in a case where the second optical fibers 21, 22, 23, 24 and 25 are pushed toward the base-end side, the flanges 21C, 22C, 23C, 24C and 25C engage the contact portion 35 so that the light-emitting end faces 21B, 22B, 23B, 24B and 25B of the second optical fibers 21, 22, 23, 24 and 25 can move toward the base-end side mutually independently without receding from the tip face 35A of the contact portion 35. If the second optical fibers 21, 22, 23, 24 and 25 should happen to stick to each other in a case where thermal tightening is applied, the second optical fibers 21, 22, 23, 24 and 25 would be peeled off each other so as to be capable of moving toward the base-end side mutually independently. In a case where thermal tightening is applied, though, the second optical fibers 21, 22, 23, 24 and 25 can be prevented from sticking to each other even though thermal tightening is applied, and each of the second optical fibers 21, 22, 23, 24 and 25 can be rendered movable toward the base-end side mutually independently by inserting spacers between the tips of the second optical fibers 21, 22, 23, 24 and 25 such that the optical fibers do not become affixed to each other and then removing these spacers after the application of thermal tightening.

It will suffice if the second optical fibers 21, 22, 23, 24 and 25 do not recede relative to the tip face 35A of the contact portion 35, and the second optical fibers 21, 22, 23, 24 and 25 may jut out from tip face 35A. An arrangement may be adopted, though, in which the second optical fibers 21, 22, 23, 24 and 25 are fixed to the tip face 35A (the tip of the contact portion 35). For example, it may be so arranged that a projection is formed on the side face of each of the second optical fibers 21, 22, 23, 24 and 25, recesses are formed on the inner wall of the contact portion 35, and the projections are fitted into the recesses. In any case, it will suffice if the tip side of each of the second optical fibers 21, 22, 23, 24 and 25 is movable independently in the optical-axis direction of the second optical fibers 21, 22, 23, 24 and 25 and the base-end side does not move.

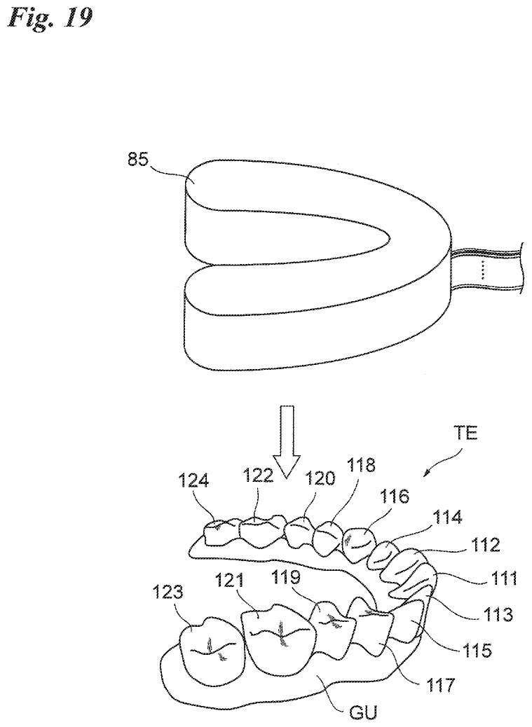

FIGS. 19 to 25, which illustrate another embodiment, show a mouthpiece 85 serving as the measurement probe. Specifically, the mouthpiece 85 corresponds to one mode of the contact portion 35 (holder).

The upper part of FIG. 19 is a perspective view of the mouthpiece 85, and the lower part a perspective view gums GU and lower teeth TE (central incisors 111 and 112, lateral incisors 113 and 114, canines 115 and 116, first premolars 117 and 118, second premolars 119 and 120, first molars 121 and 122, and second molars 123 and 124) on which the mouthpiece 85 is placed.

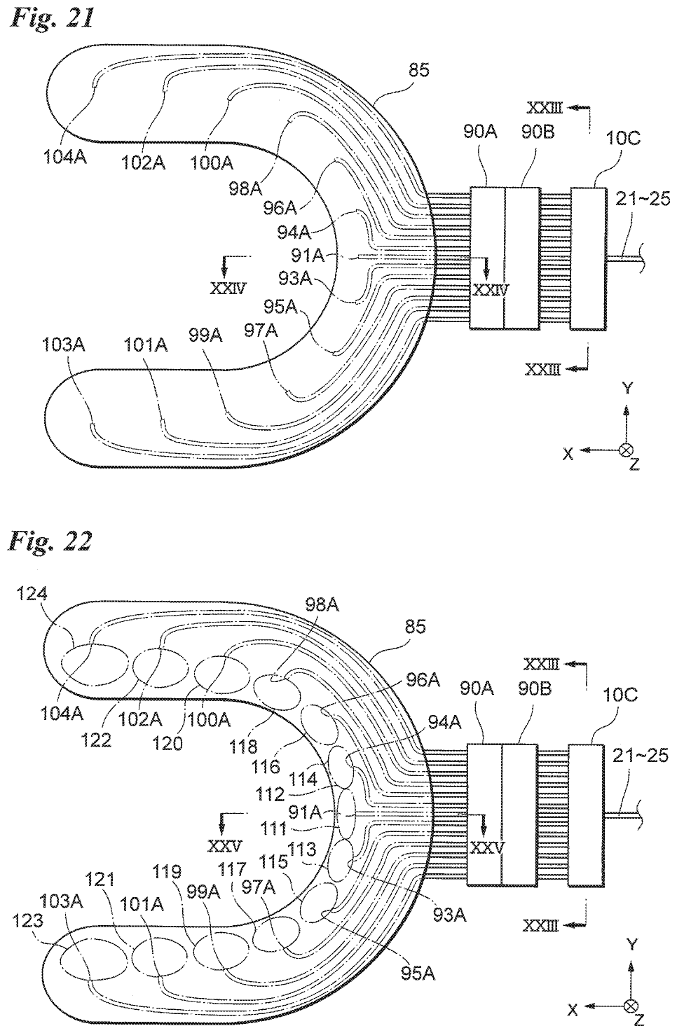

As will be described below in detail (refer to FIGS. 21, 22 and the like), the interior of the mouthpiece 85 includes a plurality of optical fibers. The mouthpiece 85 is made of the same flexible material as that of the contact portion 35 and holds the plurality of optical fibers, which are included in the mouthpiece 85, freely movable independently along the direction of the optical axes of the optical fibers.

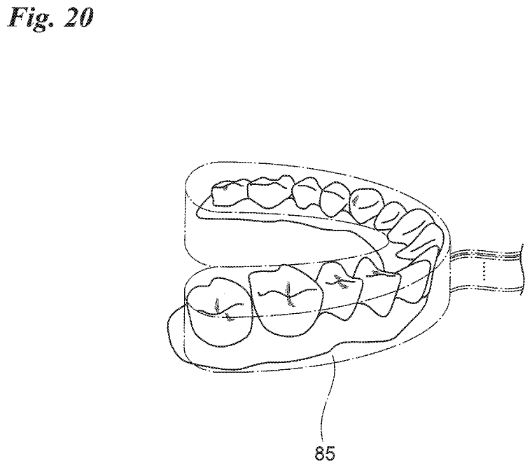

FIG. 20 illustrates the manner in which the mouthpiece 85 is placed on teeth TE and gums GU.

A space is formed inside the mouthpiece 85, and the inner surface of the mouthpiece 85 is in close contact with the surface of the teeth TE and the surface of the gums GU.

FIG. 21 is a plan view of the mouthpiece 85.

The mouthpiece 85 includes a number of optical fibers such as optical fibers 91A to 104A. The number of optical fibers extend to the exterior of the mouthpiece 85 from the front (the right side in FIG. 20) thereof. The number of optical fibers are separably coupled by a pair of connectors (connector 90A and connector 90B). That is, the connector 90A and the connector 90B, which are provided between the tip side and base-end side of a second optical waveguide array of the optical waveguide arrays, are an example of coupling members for separably coupling the second optical waveguide array.

The optical fibers such as the optical fibers 91A to 104A that extend from the connector 90B are connected to one end of the deflecting unit 10C (which has a structure identical with that of the deflecting unit 10 shown in FIG. 2 but which may have the structure of the deflecting unit 10A or the structure of the deflecting unit 10B), and the five optical fibers 21 to 25 are connected to the other end of the deflecting unit 10C. Using the deflecting mirror provided within the deflecting unit 10C, the deflecting unit 10C deflects the measuring light LM, which is emitted from optical fibers 21 to 25, causing the measuring light LM to propagate toward optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E, or 104A to 104E.

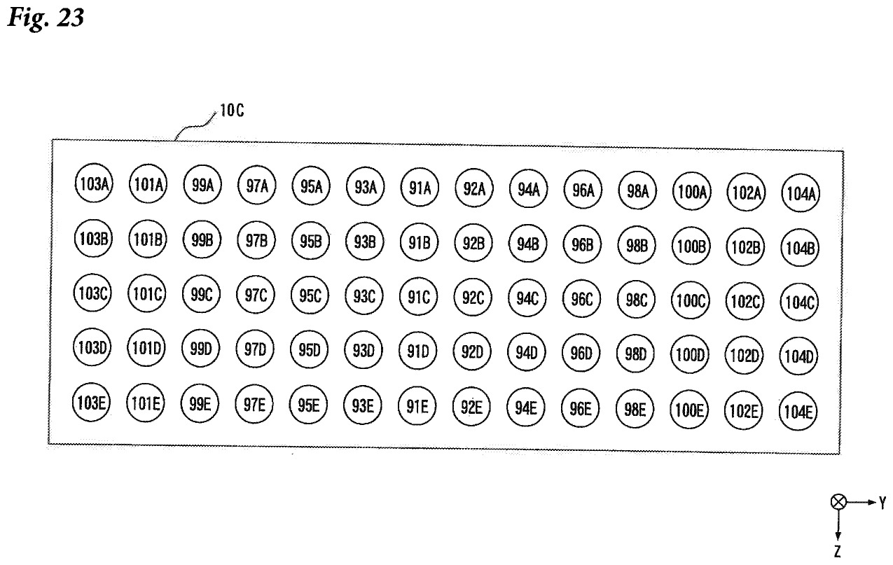

FIG. 23 is a sectional view taken along line XXIII-XXIII of FIG. 21. Hatching is omitted in FIG. 23.

The number of optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E and 104A to 104E, which extend to the outside from the mouthpiece 85, are connected to the deflecting unit 10C. The number of optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E and 104A to 104E are arrayed in respective rows along the direction of the Z-axis (up-and-down direction).

The measuring light LM emitted from among the optical fibers 21 to 25 is deflected toward the optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E, or 104A to 104E by the deflecting unit 10C. The deflecting unit 10C shown in FIGS. 21 to 23 thus deflects the measuring light LM, which has been emitted from the first fiber 7, in the direction of one dimension. However, the deflecting unit 10C may deflect the measuring light LM, which has been emitted from the optical fibers 21 to 25, in the directions of two dimensions. In FIG. 21 or 22, the first optical fiber 7, rather than the five optical fibers 21 to 25, may be connected to the deflecting unit 10C. In this case, since a deflecting unit for deflection from the first optical fiber 7 to the five optical fibers 21 to 25 will no longer be necessary, the deflecting units provided for the overall periodontal disease examination apparatus will now be a single unit.

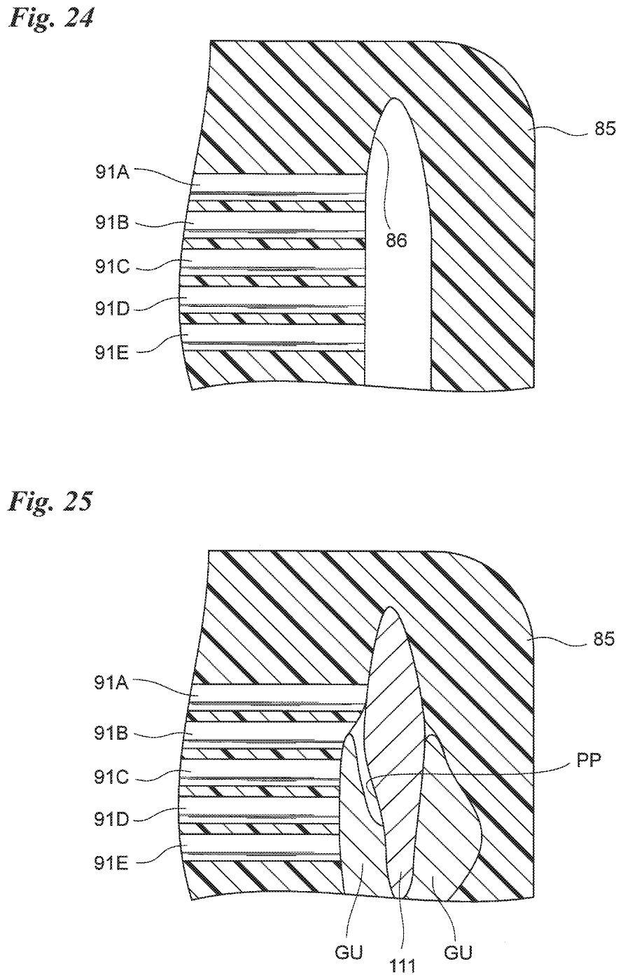

FIG. 22 is a plan view illustrating the manner in which the mouthpiece 85 is placed on the teeth TE and gums GU. FIG. 24 is a sectional view taken along line XXIV-XXIV of FIG. 21, and FIG. 25 is a sectional view taken along line XXV-XXV of FIG. 22.

The optical fibers 91A to 91E arrayed in a single row are held by the mouthpiece 85 such that the light-emitting faces of the optical fibers 91A to 91E are exposed at a close-contact surface 86 of the teeth TE and gums GU.

When the mouthpiece 85 is placed on the teeth TE and gums GU, the light-emitting faces (on the right side in FIG. 24) of the optical fibers 91A to 91E come into close contact with the surface of gum GU, which envelops the central incisor 111, and the surface of the medium incisor 111 on its outer side, as illustrated in FIGS. 22 and 24.

Similarly, when the mouthpiece 85 is placed on the teeth TE and gums GU, the light-emitting faces of the optical fibers 92A to 92E come into close contact with the surface of gum GU, which envelops the central incisor 112, and with the surface of the medium incisor 112 on its outer side, the light-emitting faces of the optical fibers 93A to 93E come into close contact with the surface of gum GU, which envelops the lateral incisor 113, and with the surface of the lateral incisor 113 on its outer side, the light-emitting faces of the optical fibers 94A to 94E come into close contact with the surface of gum GU, which envelops the with incisor 114, and with the surface of the lateral incisor 114 on its outer side, the light-emitting faces of the optical fibers 95A to 95E come into close contact with the surface of gum GU, which envelops the canine 115, and with the surface of the canine 115 on its outer side, and the light-emitting faces of the optical fibers 96A to 96E come into close contact with the surface of gum GU, which envelops the canine 116, and with the surface of the canine 116 on its outer side. Further, the light-emitting faces of the optical fibers 97A to 97E come into close contact with the surface of gum GU, which envelops the first premolar 117, and with the surface of the first premolar 117 on its outer side, the light-emitting faces of the optical fibers 98A to 98E come into close contact with the surface of gum GU, which envelops the first premolar 118, and with the surface of the first premolar 118 on its outer side, the light-emitting faces of the optical fibers 99A to 99E come into close contact with the surface of gum GU, which envelops the second premolar 119, and with the surface of the second premolar 119 on its outer side, the light-emitting faces of the optical fibers 100A to 100E come into close contact with the surface of gum GU, which envelops the second premolar 120, and with the surface of the second premolar 120 on its outer side, the light-emitting faces of the optical fibers 101A to 101E come into close contact with the surface of gum GU, which envelops the first molar 121, and with the surface of the first molar 121 on its outer side, the light-emitting faces of the optical fibers 102A to 102E come into close contact with the surface of gum GU, which envelops the first molar 122, and with the surface of the first molar 122 on its outer side, the light-emitting faces of the optical fibers 103A to 103E come into close contact with the surface of gum GU, which envelops the second molar 123, and with the surface of the second molar 123 on its outer side, and the light-emitting faces of the optical fibers 104A to 104E come into close contact with the surface of the second molar 123 on its outer side.

When the measuring light LM emitted from the second optical fibers 21 to 25 is deflected and impinges on the optical fibers 91A to 91E, the measuring light LM irradiates the gum GU, which envelops the central incisor 111, as well as the central incisor 111, and optical tomographic images of the gum GU enveloping the central incisor 111 and of the central incisor 111 are therefore obtained. When the measuring light LM emitted from the second optical fibers 21 to 25 is deflected and impinges on the optical fibers 92A to 92E, the measuring light LM irradiates the gum GU, which envelops the central incisor 112, as well as the central incisor 112, and optical tomographic images of the gum GU enveloping the central incisor 112 and of the central incisor 112 are therefore obtained. When the measuring light LM emitted from the second optical fibers 21 to 25 is deflected and impinges on the optical fibers 93A to 93E, the measuring light LM irradiates the gum GU, which envelops the lateral incisor 113, as well as the lateral incisor 113, and optical tomographic images of the gum GU enveloping the lateral incisor 113 and of the lateral incisor 113 are therefore obtained. When the measuring light LM emitted from the second optical fibers 21 to 25 is deflected and impinges on the optical fibers 94A to 94E, the measuring light LM irradiates the gum GU, which envelops the lateral incisor 114, as well as the lateral incisor 114, and optical tomographic images of the gum GU enveloping the lateral incisor 114 and of the lateral incisor 114 are therefore obtained. When the measuring light LM emitted from the second optical fibers 21 to 25 is deflected and impinges on the optical fibers 95A to 95E, the measuring light LM irradiates the gum GU, which envelops the canine 115, as well as the canine 115, and optical tomographic images of the gum GU enveloping the canine 115 and of the canine 115 are therefore obtained. When the measuring light LM emitted from the second optical fibers 21 to 25 is deflected and impinges on the optical fibers 96A to 96E, the measuring light LM irradiates the gum GU, which envelops the canine 116, as well as the canine 116, and optical tomographic images of the gum GU enveloping the canine 116 and of the canine 116 are therefore obtained.

Similarly, when the measuring light LM emitted from the second optical fibers 21 to 25 is deflected and impinges on the optical fibers 97A to 97E, the measuring light LM irradiates the gum GU, which envelops the first premolar 117, as well as the first premolar 117, and optical tomographic images of the gum GU enveloping the first premolar 117 and of the first premolar 117 are therefore obtained. When the measuring light LM emitted from the second optical fibers 21 to 25 is deflected and impinges on the optical fibers 98A to 98E, the measuring light LM irradiates the gum GU, which envelops the first premolar 118, as well as the first premolar 118, and optical tomographic images of the gum GU enveloping the first premolar 118 and of the first premolar 118 are therefore obtained. When the measuring light LM emitted from the second optical fibers 21 to 25 is deflected and impinges on the optical fibers 99A to 99E, the measuring light LM irradiates the gum GU, which envelops the second premolar 119, as well as the second premolar 119, and optical tomographic images of the gum GU enveloping the second premolar 119 and of the second premolar 119 are therefore obtained. When the measuring light LM emitted from the second optical fibers 21 to 25 is deflected and impinges on the optical fibers 100A to 100E, the measuring light LM irradiates the gum GU, which envelops the second premolar 120, as well as the second premolar 120, and optical tomographic images of the gum GU enveloping the second premolar 120 and of the second premolar 120 are therefore obtained.

Furthermore, when the measuring light LM emitted from the second optical fibers 21 to 25 is deflected and impinges on the optical fibers 101A to 101E, the measuring light LM irradiates the gum GU, which envelops the first molar 121, as well as the first molar 121, and optical tomographic images of the gum GU enveloping the first molar 121 and of the first molar 121 are therefore obtained. When the measuring light LM emitted from the second optical fibers 21 to 25 is deflected and impinges on the optical fibers 102A to 102E, the measuring light LM irradiates the gum GU, which envelops the first molar 122, as well as the first molar 122, and optical tomographic images of the gum GU enveloping the first molar 122 and of the first molar 122 are therefore obtained. When the measuring light LM emitted from the second optical fibers 21 to 25 is deflected and impinges on the optical fibers 103A to 103E, the measuring light LM irradiates the gum GU, which envelops the second molar 123, as well as the second molar 123, and optical tomographic images of the gum GU enveloping the second molar 123 and of the second molar 123 are therefore obtained. When the measuring light LM emitted from the second optical fibers 21 to 25 is deflected and impinges on the optical fibers 104A to 104E, the measuring light LM irradiates the gum GU, which envelops the second molar 124, as well as the second molar 124, and optical tomographic images of the gum GU enveloping the second molar 124 and of the second molar 124 are therefore obtained.

By placing the mouthpiece 85 on the teeth TE and gums GU and causing the measuring light LM to propagate toward the second optical fibers 21 to 25, the measurer can detect the depths of multiple periodontal pockets corresponding to multiple teeth TE without manually performing alignment successively with respect to each tooth TO undergoing measurement and the gum containing each tooth TO. As a result, in comparison with a case where the measurer performs such alignment successively with respect to each periodontal pocket corresponding to each tooth TO, it is possible to achieve a reduction in measurer inconvenience and a shortening of measurement time.

In the foregoing embodiment, since the multiplicity of optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E and 104A to 104E are each held by the mouthpiece 85 made of a flexible material, they are free to move independently of each other along the direction of the optical axis. The above-described mouthpiece 85 can be produced by introducing the multiplicity of optical fibers into a previously prepared mold of the mouthpiece 85 and pouring in a resin of flexible material. Alternatively, the above-described mouthpiece 85 may be produced by molding the shape of the mouthpiece 85 using a resin of flexible material, thereafter forming the above-described space portions (the space portion 32 of gripping portion 31 and the space portion 36 of contact portion 35), and passing the multiplicity of optical fibers through these space portions. Further, regardless of which method of production is used, preferably a GRIN lens is provided on the tip of each of the multiplicity of optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E and 104A to 104E, or the tip of each of the multiplicity of optical fibers 91A to 91E, 92A to 92E, 93A to 93E, 94A to 94E, 95A to 95E, 96A to 96E, 97A to 97E, 98A to 98E, 99A to 99E, 100A to 100E, 101A to 101E, 102A to 102E, 103A to 103E and 104A to 104E is machined to make it a parallelizing element.

In the foregoing embodiment, the mouthpiece 85 for the lower jaw is described. However, the depths of periodontal pockets can be detected in similar fashion with a mouthpiece 85 for the upper jaw rather than the lower jaw. Further, it may be so arranged as to provide optical fibers inside the mouthpiece 85 so as to detect the depths of a periodontal pockets on the inner-side surface of the teeth TE rather than the depths of a periodontal pockets on the outer-side surface of the teeth TE. In such case the optical fibers would be provided inside the mouthpiece 85 such that their light-emitting faces will come into contact with inner-side surface of the teeth TE. Furthermore, in the foregoing embodiment, it is so arranged that the light-emitting end faces of the optical fibers of one row come into contact with one tooth. However, it may be so arranged that the light-emitting end faces of the optical fibers of two or more rows come into contact with one tooth.

In the foregoing embodiment, as illustrated in FIGS. 21 and 22, the detachable connectors 90A and 90B are provided between the tip side and base-end side of the second optical waveguide array of the optical waveguide arrays so as to separably couple the second optical waveguide array. However, the provision of connecting members naturally is not limited to a case where an optical waveguide array includes a first optical waveguide array and a second optical waveguide array. That is, connectors (one example of a coupling member) may be provided between the tips of the optical fibers 21 to 25, which are shown in figures such as FIG. 1, and the base ends of the optical fibers 21 to 25 for separably coupling each of the optical fibers 21 to 25. In any case, the examination probe 30 can be detached from the base ends of the optical fibers and the examination probe 30 can be replaced comparatively simply. As a result, owing to the fact that it is possible to detach the part that includes the tip of the optical fiber array which comes into contact with the oral cavity of the subject, it is possible to discard this part and replace it with an unused part on a per-subject basis. The degree of hygiene in examination of periodontal disease, therefore, can be improved. Furthermore, by virtue of the fact that the discarded portion does not include comparatively expensive members such as the deflecting unit, running cost is reduced in comparison with a case where these members are included in the discarded portion.

1. A periodontal disease examination apparatus comprising:
an optical divider for splitting low-interference light into measuring light and reference light;
a first optical waveguide on which the measuring light split off by said optical divider is incident;
an optical waveguide array in which a plurality of second optical waveguides are arrayed in at least a single row;
a holder formed of a flexible member for holding said optical waveguide array such that each of the plurality of second optical waveguides is freely movable independently along the direction of its own optical axis;
a first control mechanism for controlling the measuring light, which is emitted from said first optical waveguide, said first optical waveguide, and at least one of the second optical waveguides in such a manner that the measuring light emitted from said first optical waveguide impinges successively on each of the second optical waveguides constituting said optical waveguide array;
a photodetector for detecting reflected light and outputting an interference signal, the reflected light being reflected measuring light which is reflected from a gum or tooth owing to irradiation of the gum or tooth with the measuring light emitted from said optical waveguide array and reflected reference light which is split off by said optical divider and reflected by a reference surface; and
a periodontal pocket data generating device for generating data regarding depth of a periodontal pocket based on the interference signal output from said photodetector.

2. A periodontal disease examination apparatus according to claim 1, wherein said holder holds each second optical waveguide constituting the optical waveguide array such that the tip of each second optical waveguide constituting said optical waveguide array is protrudable from the tip of said holder but incapable of receding into the tip of said holder.

3. A periodontal disease examination apparatus according to claim 1, wherein said holder has a space portion;
each second optical waveguide constituting said optical waveguide array passes through said space portion; and
the diameter of the tip of each second optical waveguide constituting said optical waveguide array is larger than the diameter of the portion of said second optical waveguide other than the tip thereof and larger than the diameter of said space portion.

4. A periodontal disease examination apparatus according to claim 1, wherein said holder fixes the tip of each second optical waveguide constituting said optical waveguide array to the tip of said holder.

5. A periodontal disease examination apparatus according to claim 1, wherein said first control mechanism is a first adjusting mechanism for adjusting at least one of: position of a measuring-light-emitting end face of said first optical waveguide, and positions of measuring-light-incident end faces of the second optical waveguides, in such a manner that the measuring-light-emitting end face of said first optical waveguide successively opposes the measuring-light-incident end faces of the second optical waveguides constituting said optical waveguide array.

6. A periodontal disease examination apparatus according to claim 5, further comprising a parallelizing element, which is for parallelizing the measuring light, provided on the optical path of the measuring light between the measuring-light-incident end faces of the second optical waveguides and the measuring-light-emitting end face of said first optical waveguide.

7. A periodontal disease examination apparatus according to claim 5, wherein said optical waveguide array includes a first optical waveguide array and a second optical waveguide array;
said first optical waveguide array includes a plurality of optical waveguides arrayed in at least one row, each of the plurality of these optical waveguides receiving incident light from its measuring-light-incident end face emitted from said first optical waveguide and emitting the measuring light from its measuring-light-emitting end face;
said second optical waveguide array includes a plurality of optical waveguides arrayed in at least one row and of a number greater than the number of the plurality of optical waveguides included in said first optical waveguide array, each of the plurality of these optical waveguides receiving incident light from its measuring-light-incident end face emitted from an optical waveguide included in said first optical waveguide array and emitting the measuring light from its measuring-light-emitting end face;
said holder holds the plurality of optical waveguides such that each of the plurality of second optical waveguides included in said second optical waveguide array is freely movable independently along the direction of its own optical axis; and
the apparatus further comprises a second control mechanism for controlling at least one of: optical waveguides included in said first optical waveguide array, and optical waveguides included in said second optical waveguide array, in such a manner that measuring light emitted from an optical waveguide included in the first optical waveguide array impinges successively on the plurality of optical waveguides included in said second optical waveguide array.

8. A periodontal disease examination apparatus according to claim 7, further comprising a parallelizing element, which is for parallelizing the measuring light, provided on the optical path of the measuring light between at least one of the following: between the measuring-light-incident end faces of the optical waveguides included in said first optical waveguide array and the measuring-light-emitting end face of said first optical waveguide, and between the measuring-light-incident end faces of the optical waveguides included in said second optical waveguide array and the measuring-light-emitting end faces of the optical waveguides included in said first optical waveguide array.

9. A periodontal disease examination apparatus according to claim 1, wherein said first control mechanism is a deflecting mechanism for deflecting measuring light emitted from said first optical waveguide and guiding the measuring light successively to each of the second optical waveguides constituting said optical waveguide array.

10. A periodontal disease examination apparatus according to claim 1, wherein a coupling member for separably coupling said optical waveguide array is provided between the tip of said optical waveguide array and the base end of said optical waveguide array.

11. A periodontal disease examination apparatus according to claim 1, wherein said holder for holding said optical waveguide array is a mouthpiece for being placed in close contact with a surface portion of teeth at a gum boundary and with a portion of the gums;
the light-emitting end faces of the plurality of second optical waveguides being exposed at a surface of close contact of the mouthpiece in close contact with the surface portion of the teeth and the portion of the gums.

12. An optical waveguide array having a plurality of optical waveguides arrayed in at least a single row, wherein out of measuring light and reference light split by an optical divider, the measuring light impinges successively on the optical waveguides, each of the optical waveguides being held freely movable independently along the direction of its own optical axis by a holder comprising a flexible material.

13. A mouthpiece placed in close contact with a surface portion of teeth at a gum boundary and with a portion of the gums and comprising a flexible material, said mouthpiece holding a plurality of optical waveguides such that light-emitting end faces of the plurality of optical waveguides arrayed in at least a single row are exposed at a surface of close contact where said mouthpiece is in close contact with the surface portion of the teeth and the portion of the gums.
